SEQUENCE LISTING (46 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - DNA, 1083 bp, Homo sapiens
  source 1..1083: mol_type = genomic DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 3 - protein, 1682 aa, Homo sapiens
MHRAVDPPGARAAREAFALGGLSCAGAWSSCPPHPPPRSAWLPGGRCSASIGQPPLPAPLPPSHGSSSGHPSKPYYAPGAPTPRPLHGKLESLHGCVQALLREPAQPGLWEQLGQLYESEHDSEEATRCYHSALRYGGSFAELGPRIGRLQQAQLWNFHTGSCQHRAKVLPPLEQVWNLLHLEHKRNYGAKRGGPPVKRAAEPPVVQPVPPAALSGPSGEEGLSPGGKRRRGCNSEQTGLPPGLPLPPPPLPPPPPPPPPPPPPLPGLATSPPFQLTKPGLWSTLHGDAWGPERKGSAPPERQEQRHSLPHPYPYPAPAYTAHPPGHRLVPAAPPGPGPRPPGAESHGCLPATRPPGSDLRESRVQRSRMDSSVSPAATTACVPYAPSRPPGLPGTTTSSSSSSSSNTGLRGVEPNPGIPGADHYQTPALEVSHHGRLGPSAHSSRKPFLGAPAATPHLSLPPGPSSPPPPPCPRLLRPPPPPAWLKGPACRAAREDGEILEELFFGTEGPPRPAPPPLPHREGFLGPPASRFSVGTQDSHTPPTPPTPTTSSSNSNSGSHSSSPAGPVSFPPPPYLARSIDPLPRPPSPAQNPQDPPLVPLTLALPPAPPSSCHQNTSGSFRRPESPRPRVSFPKTPEVGPGPPPGPLSKAPQPVPPGVGELPARGPRLFDFPPTPLEDQFEEPAEFKILPDGLANIMKMLDESIRKEEEQQQHEAGVAPQPPLKEPFASLQSPFPTDTAPTTTAPAVAVTTTTTTTTTTTATQEEEKKPPPALPPPPPLAKFPPPSQPQPPPPPPPSPASLLKSLASVLEGQKYCYRGTGAAVSTRPGPLPTTQYSPGPPSGATALPPTSAAPSAQGSPQPSASSSSQFSTSGGPWARERRAGEEPVPGPMTPTQPPPPLSLPPARSESEVLEEISRACETLVERVGRSATDPADPVDTAEPADSGTERLLPPAQAKEEAGGVAAVSGSCKRRQKEHQKEHRRHRRACKDSVGRRPREGRAKAKAKVPKEKSRRVLGNLDLQSEEIQGREKSRPDLGGASKAKPPTAPAPPSAPAPSAQPTPPSASVPGKKAREEAPGPPGVSRADMLKLRSLSEGPPKELKIRLIKVESGDKETFIASEVEERRLRMADLTISHCAADVVRASRNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEEKESEDEESEEPDSTTGTPPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLVRARRARGQRRRALGQAAGTGFGSPAAPFPEPPPAFSPQAPASTSR

SEQ ID NO: 4 - protein, 659 aa, Homo sapiens
QSEEIQGREKSRPDLGGASKAKPPTAPAPPSAPAPSAQPTPPSASVPGKKAREEAPGPPGVSRADMLKLRSLSEGPPKELKIRLIKVESGDKETFIASEVEERRLRMADLTISHCAADVVRASRNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEEKESEDEESEEPDSTTGTPPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLVRARRARGQRRRALGQAAGTGFGSPAAPFPEPPPAFSPQAPASTSR

SEQ ID NO: 5 - protein, 109 aa, Homo sapiens
QLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNV

SEQ ID NO: 6 - DNA, 5049 bp, Homo sapiens
  source 1..5049: mol_type = genomic DNA
atgcatcgggcagtggatcctccaggggcccgcgctgcacgggaagcctttgcccttgggggcctgagctgtgctggggcctggagctcctgcccgcctcatccccctcctcgtagcgcatggctgcctggaggcagatgctcagccagcattgggcagcccccgcttcctgctcccctacccccttcacatggcagtagttctgggcaccccagcaaaccatattatgctccaggggcgcccactccaagacccctccatgggaagctggaatccctgcatggctgtgtgcaggcattgctccgggagccagcccagccagggctttgggaacagcttgggcaactgtacgagtcagagcacgatagtgaggaggccacacgctgctaccacagcgcccttcgatacggaggaagcttcgctgagctggggccccgcattggccgactgcagcaggcccagctctggaactttcatactggctcctgccagcaccgagccaaggtcctgcccccactggagcaagtgtggaacttgctacaccttgagcacaaacggaactatggagccaagcggggaggtcccccggtgaagcgagctgctgaacccccagtggtgcagcctgtgcctcctgcagcactctcaggcccctcaggggaggagggcctcagccctggaggcaagcgaaggagaggctgcaactctgaacagactggccttcccccagggctgccactgcctccaccaccattaccaccaccaccaccaccaccaccaccaccaccaccacccctgcctggcctggctaccagccccccatttcagctaaccaagccagggctgtggagtaccctgcatggagatgcctggggcccagagcgcaagggttcagcacccccagagcgccaggagcagcggcactcgctgcctcacccatatccatacccagctccagcgtacaccgcgcacccccctggccaccggctggtcccggctgctcccccaggcccaggcccccgccccccaggagcagagagccatggctgcctgcctgccacccgtccccccggaagtgaccttagagagagcagagttcagaggtcgcggatggactccagcgtttcaccagcagcaaccaccgcctgcgtgccttacgccccttcccggccccctggcctccccggcaccaccaccagcagcagcagtagcagcagcagcaacactggtctccggggcgtggagccgaacccaggcattcccggcgctgaccattaccaaactcccgcgctggaggtctctcaccatggccgcctggggccctcggcacacagcagtcggaaaccgttcttgggggctcccgctgccactccccacctatccctgccacctggaccttcctcaccccctccacccccctgtccccgcctcttacgccccccaccaccccctgcctggttgaagggtccggcctgccgggcagcccgagaggatggagagatcttagaagagctcttctttgggactgagggacccccccgccctgccccaccacccctcccccatcgcgagggcttcttggggcctccggcctcccgcttttctgtgggcactcaggattctcacacccctcccactcccccaaccccaaccaccagcagtagcaacagcaacagtggcagccacagcagcagccctgctgggcctgtgtcctttcccccaccaccctatctggccagaagtatagacccccttccccggcctcccagcccagcacagaacccccaggacccacctcttgtacccctgactcttgccctgcctccagcccctccttcctcctgccaccaaaatacctcaggaagcttcaggcgcccggagagcccccggcccagggtctccttcccaaagacccccgaggtggggccggggccacccccaggccccctgagtaaagccccccagcctgtgccgcccggggttggggagctgcctgcccgaggccctcgactctttgattttccccccactccgctggaggaccagtttgaggagccagccgaattcaagatcctacctgatgggctggccaacatcatgaagatgctggacgaatccattcgcaaggaagaggaacagcaacaacacgaagcaggcgtggccccccaacccccgctgaaggagccctttgcatctctgcagtctcctttccccaccgacacagcccccaccactactgctcctgctgtcgccgtcaccaccaccaccaccaccaccaccaccaccacggccacccaggaagaggagaagaagccaccaccagccctaccaccaccaccgcctctagccaagttccctccaccctctcagccacagccaccaccacccccaccccccagcccggccagcctgctcaaatccttggcctccgtgctggagggacaaaagtactgttatcgggggactggagcagctgtttccacccggcctgggcccttgcccaccactcagtattcccctggccccccatcaggtgctaccgccctgccgcccacctcagcggcccctagcgcccagggctccccacagccctctgcttcctcgtcatctcagttctctacctcaggcgggccctgggcccgggagcgcagggcgggcgaagagccagtcccgggccccatgacccccacccaaccgcccccacccctatctctgccccctgctcgctctgagtctgaggtgctagaagagatcagccgggcttgcgagacccttgtggagcgggtgggccggagtgccactgacccagccgacccagtggacacagcagagccagcggacagtgggactgagcgactgctgccccccgcacaggccaaggaggaggctggcggggtggcggcagtgtcaggcagctgtaagcggcgacagaaggagcatcagaaggagcatcggcggcacaggcgggcctgtaaggacagtgtgggtcgtcggccccgtgagggcagggcaaaggccaaggccaaggtccccaaagaaaagagccgccgggtgctggggaacctggacctgcagagcgaggagatccagggtcgtgagaagtcccggcccgatcttggcggggcctccaaggccaagccacccacagctccagcccctccatcagctcctgcaccttctgcccagcccacacccccgtcagcctctgtccctggaaagaaggctcgggaggaagccccagggccaccgggtgtcagccgggccgacatgctgaagctgcgctcacttagtgaggggccccccaaggagctgaagatccggctcatcaaggtagagagtggtgacaaggagacctttatcgcctctgaggtggaagagcggcggctgcgcatggcagacctcaccatcagccactgtgctgctgacgtcgtgcgcgccagcaggaatgccaaggtgaaagggaagtttcgagagtcctacctttcccctgcccagtctgtgaaaccgaagatcaacactgaggagaagctgccccgggaaaaactcaacccccctacacccagcatctatctggagagcaaacgggatgccttctcacctgtcctgctgcagttctgtacagaccctcgaaatcccatcacagtgatccggggcctggcgggctccctgcggctcaacttgggcctcttctccaccaagaccctggtggaagcgagtggcgaacacaccgtggaagttcgcacccaggtgcagcagccctcagatgagaactgggatctgacaggcactcggcagatctggccttgtgagagctcccgttcccacaccaccattgccaagtacgcacagtaccaggcctcatccttccaggagtctctgcaggaggagaaggagagtgaggatgaggagtcagaggagccagacagcaccactggaacccctcctagcagcgcaccagacccgaagaaccatcacatcatcaagtttggcaccaacatcgacttgtctgatgctaagcggtggaagccccagctgcaggagctgctgaagctgcccgccttcatgcgggtaacatccacgggcaacatgctgagccacgtgggccacaccatcctgggcatgaacacggtgcagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtggggcccctcaccgcctatcagtaccagctggccctggaacgatacgagtggaatgaggtgaagaacgtcaaatccatcgtgcccatgattcacgtgtcatggaacgtggctcgcacggtcaaaatcagcgaccccgacttgttcaagatgatcaagttctgcctgctgcagtccatgaagcactgccaggtgcaacgcgagagcctggtgcgggcagggaagaaaatcgcttaccagggccgtgtcaaggacgagccagcctactactgcaacgagtgcgatgtggaggtgtttaacatcctgttcgtgacaagtgagaatggcagccgcaacacgtacctggtacactgcgagggctgtgcccggcgccgcagcgcaggcctgcagggcgtggtggtgctggagcagtaccgcactgaggagctggctcaggcctacgacgccttcacgctggtgagggcccggcgggcgcgcgggcagcggaggagggcactggggcaggctgcagggacgggcttcgggagcccggccgcgcctttccctgagcccccgccggctttctccccccaggccccagccagcacgtcgcgatga

SEQ ID NO: 7 - DNA, 1980 bp, Homo sapiens
  source 1..1980: mol_type = genomic DNA
cagagcgaggagatccagggtcgtgagaagtcccggcccgatcttggcggggcctccaaggccaagccacccacagctccagcccctccatcagctcctgcaccttctgcccagcccacacccccgtcagcctctgtccctggaaagaaggctcgggaggaagccccagggccaccgggtgtcagccgggccgacatgctgaagctgcgctcacttagtgaggggccccccaaggagctgaagatccggctcatcaaggtagagagtggtgacaaggagacctttatcgcctctgaggtggaagagcggcggctgcgcatggcagacctcaccatcagccactgtgctgctgacgtcgtgcgcgccagcaggaatgccaaggtgaaagggaagtttcgagagtcctacctttcccctgcccagtctgtgaaaccgaagatcaacactgaggagaagctgccccgggaaaaactcaacccccctacacccagcatctatctggagagcaaacgggatgccttctcacctgtcctgctgcagttctgtacagaccctcgaaatcccatcacagtgatccggggcctggcgggctccctgcggctcaacttgggcctcttctccaccaagaccctggtggaagcgagtggcgaacacaccgtggaagttcgcacccaggtgcagcagccctcagatgagaactgggatctgacaggcactcggcagatctggccttgtgagagctcccgttcccacaccaccattgccaagtacgcacagtaccaggcctcatccttccaggagtctctgcaggaggagaaggagagtgaggatgaggagtcagaggagccagacagcaccactggaacccctcctagcagcgcaccagacccgaagaaccatcacatcatcaagtttggcaccaacatcgacttgtctgatgctaagcggtggaagccccagctgcaggagctgctgaagctgcccgccttcatgcgggtaacatccacgggcaacatgctgagccacgtgggccacaccatcctgggcatgaacacggtgcagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtggggcccctcaccgcctatcagtaccagctggccctggaacgatacgagtggaatgaggtgaagaacgtcaaatccatcgtgcccatgattcacgtgtcatggaacgtggctcgcacggtcaaaatcagcgaccccgacttgttcaagatgatcaagttctgcctgctgcagtccatgaagcactgccaggtgcaacgcgagagcctggtgcgggcagggaagaaaatcgcttaccagggccgtgtcaaggacgagccagcctactactgcaacgagtgcgatgtggaggtgtttaacatcctgttcgtgacaagtgagaatggcagccgcaacacgtacctggtacactgcgagggctgtgcccggcgccgcagcgcaggcctgcagggcgtggtggtgctggagcagtaccgcactgaggagctggctcaggcctacgacgccttcacgctggtgagggcccggcgggcgcgcgggcagcggaggagggcactggggcaggctgcagggacgggcttcgggagcccggccgcgcctttccctgagcccccgccggctttctccccccaggccccagccagcacgtcgcgatga

SEQ ID NO: 8 - DNA, 327 bp, Homo sapiens
  source 1..327: mol_type = genomic DNA
cagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtg

SEQ ID NO: 9 - protein, 12 aa, Human immunodeficiency virus
GRKKRRQRRRPQ

SEQ ID NO: 10 - protein, 8 aa, synthetic construct
  REGION 1..8: note = r8
RRRRRRRR

SEQ ID NO: 11 - protein, 21 aa, synthetic construct
  REGION 1..21: note = MPG-8 (The first amino acid "Ala" is modified into "bAla.")
AFLGWLGAWGTMGWSPKKKRK

SEQ ID NO: 12 - DNA, 21 bp, synthetic construct
  misc_feature 1..21: note = sense strand of siRNA against POU5F1
  source 1..21: mol_type = other DNA
gcccgaaagagaaagcgaatt

SEQ ID NO: 13 - DNA, 21 bp, synthetic construct
  misc_feature 1..21: note = antisense strand of siRNA against POU5F1
  source 1..21: mol_type = other DNA
ttcgctttctctttcgggcct

SEQ ID NO: 14 - DNA, 20 bp, synthetic construct
  misc_feature 1..20: note = qRT-PCR forward primer for MYOG
  source 1..20: mol_type = other DNA
gccagactatccccttcctc

SEQ ID NO: 15 - DNA, 20 bp, synthetic construct
  misc_feature 1..20: note = qRT-PCR reverse primer for MYOG
  source 1..20: mol_type = other DNA
gaggccgcgttatgataaaa

SEQ ID NO: 16 - DNA, 18 bp, synthetic construct
  misc_feature 1..18: note = qRT-PCR forward primer for AFP
  source 1..18: mol_type = other DNA
tgggacccgaactttcca

SEQ ID NO: 17 - DNA, 22 bp, synthetic construct
  misc_feature 1..22: note = qRT-PCR reverse primer for AFP
  source 1..22: mol_type = other DNA
ggccacatccaggactagtttc

SEQ ID NO: 18 - DNA, 20 bp, synthetic construct
  misc_feature 1..20: note = qRT-PCR forward primer for COL1A1
  source 1..20: mol_type = other DNA
cctggatgccatcaaagtct

SEQ ID NO: 19 - DNA, 20 bp, synthetic construct
  misc_feature 1..20: note = qRT-PCR reverse primer for COL1A1
  source 1..20: mol_type = other DNA
tcttgtccttggggttcttg

SEQ ID NO: 20 - DNA, 20 bp, synthetic construct
  misc_feature 1..20: note = qRT-PCR forward primer for COL2A1
  source 1..20: mol_type = other DNA
tttcccaggtcaagatggtc

SEQ ID NO: 21 - DNA, 20 bp, synthetic construct
  misc_feature 1..20: note = qRT-PCR reverse primer for COL2A1
  source 1..20: mol_type = other DNA
cttcagcacctgtctcacca

SEQ ID NO: 22 - DNA, 20 bp, synthetic construct
  misc_feature 1..20: note = qRT-PCR forward primer for CD45
  source 1..20: mol_type = other DNA
tcctggactcccaaaatctg

SEQ ID NO: 23 - DNA, 20 bp, synthetic construct
  misc_feature 1..20: note = qRT-PCR reverse primer for CD45
  source 1..20: mol_type = other DNA
accttgaacccgaacatgag

SEQ ID NO: 24 - DNA, 23 bp, synthetic construct
  misc_feature 1..23: note = qRT-PCR forward primer for NESTIN
  source 1..23: mol_type = other DNA
tggttttccagagtcttcagtga

SEQ ID NO: 25 - DNA, 21 bp, synthetic construct
  misc_feature 1..21: note = qRT-PCR reverse primer for NESTIN
  source 1..21: mol_type = other DNA
gaaacagccatagagggcaaa

SEQ ID NO: 26 - DNA, 960 bp, Homo sapiens
  source 1..960: mol_type = genomic DNA
atggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctc

SEQ ID NO: 27 - DNA, 1896 bp, Homo sapiens
  source 1..1896: mol_type = genomic DNA
atggtttctaaactgagccagctgcagacggagctcctggcggccctgctggagtcagggctgagcaaagaggcactgctccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacaggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagttg

SEQ ID NO: 28 - DNA, 1521 bp, Homo sapiens
  source 1..1521: mol_type = genomic DNA
atgcgtattcccgtagatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcagatgatgacactgccacctctgacttctgcctctggccttccactctcagtaagaagagccaggcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatat

SEQ ID NO: 29 - DNA, 1527 bp, Homo sapiens
  source 1..1527: mol_type = genomic DNA
atgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgacct

SEQ ID NO: 30 - DNA, 792 bp, Homo sapiens
  source 1..792: mol_type = genomic DNA
atggaagggtttcccctcgtcccccctccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccac

SEQ ID NO: 31 - DNA, 708 bp, Homo sapiens
  source 1..708: mol_type = genomic DNA
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttc

SEQ ID NO: 32 - DNA, 2541 bp, Homo sapiens
  source 1..2541: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggcttagccaccatgtacccatacgatgttccagattacgctcctaagaaaaagaggaaggtgcagagcgaggagatccagggtcgtgagaagtcccggcccgatcttggcggggcctccaaggccaagccacccacagctccagcccctccatcagctcctgcaccttctgcccagcccacacccccgtcagcctctgtccctggaaagaaggctcgggaggaagccccagggccaccgggtgtcagccgggccgacatgctgaagctgcgctcacttagtgaggggccccccaaggagctgaagatccggctcatcaaggtagagagtggtgacaaggagacctttatcgcctctgaggtggaagagcggcggctgcgcatggcagacctcaccatcagccactgtgctgctgacgtcgtgcgcgccagcaggaatgccaaggtgaaagggaagtttcgagagtcctacctttcccctgcccagtctgtgaaaccgaagatcaacactgaggagaagctgccccgggaaaaactcaacccccctacacccagcatctatctggagagcaaacgggatgccttctcacctgtcctgctgcagttctgtacagaccctcgaaatcccatcacagtgatccggggcctggcgggctccctgcggctcaacttgggcctcttctccaccaagaccctggtggaagcgagtggcgaacacaccgtggaagttcgcacccaggtgcagcagccctcagatgagaactgggatctgacaggcactcggcagatctggccttgtgagagctcccgttcccacaccaccattgccaagtacgcacagtaccaggcctcatccttccaggagtctctgcaggaggagaaggagagtgaggatgaggagtcagaggagccagacagcaccactggaacccctcctagcagcgcaccagacccgaagaaccatcacatcatcaagtttggcaccaacatcgacttgtctgatgctaagcggtggaagccccagctgcaggagctgctgaagctgcccgccttcatgcgggtaacatccacgggcaacatgctgagccacgtgggccacaccatcctgggcatgaacacggtgcagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtggggcccctcaccgcctatcagtaccagctggccctggaacgatacgagtggaatgaggtgaagaacgtcaaatccatcgtgcccatgattcacgtgtcatggaacgtggctcgcacggtcaaaatcagcgaccccgacttgttcaagatgatcaagttctgcctgctgcagtccatgaagcactgccaggtgcaacgcgagagcctggtgcgggcagggaagaaaatcgcttaccagggccgtgtcaaggacgagccagcctactactgcaacgagtgcgatgtggaggtgtttaacatcctgttcgtgacaagtgagaatggcagccgcaacacgtacctggtacactgcgagggctgtgcccggcgccgcagcgcaggcctgcagggcgtggtggtgctggagcagtaccgcactgaggagctggctcaggcctacgacgccttcacgctggtgagggcccggcgggcgcgcgggcagcggaggagggcactggggcaggctgcagggacgggcttcgggagcccggccgcgcctttccctgagcccccgccggctttctccccccaggccccagccagcacgtcgcgatgaacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - DNA, 1574 bp, Homo sapiens
  source 1..1574: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccgcggccgcccccttcaccgctagggataacagggtaatagaaggagccgccaccatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgagtttcctgtgaacaattgctcctctcttaaggtagcaaagggtgggcgcgccgacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 34 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 35 - DNA, 2414 bp, Homo sapiens
  source 1..2414: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccaccatggtttctaaactgagccagctgcagacggagctcctggcggccctgctggagtcagggctgagcaaagaggcactgctccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacaggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccggcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagttgtgagcggccgcacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 632 aa, Homo sapiens
MVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQL

SEQ ID NO: 37 - DNA, 1222 bp, Homo sapiens
  source 1..1222: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccaccatgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggggcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccactaggacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - protein, 237 aa, Homo sapiens
MPARLETCISDLDCASSSGSDLSGFLTDEEDCARLQQAASASGPPAPARRGAPNISRASEVPGAQDDEQERRRRRGRTRVRSEALLHSLRRSRRVKANDRERNRMHNLNAALDALRSVLPSFPDDTKLTKIETLRFAYNYIWALAETLRLADQGLPGGGARERLLPPQCVPCLPGPPSPASDAESWGSGAAAASPLSDPSSPAASEDFTYRPGDPVFSFPSLPKDLLHTTPCFIPYH

SEQ ID NO: 39 - DNA, 1357 bp, Homo sapiens
  source 1..1357: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccgcggccgcccccttcaccatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggtggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctagaagggtgggcgcgccgacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - protein, 272 aa, Homo sapiens
MFVKSETLELKEEEDVLVLLGSASPALAALTPLSSSADEEEEEEPGASGGARRQRGAEAGQGARGGVAAGAEGCRPARLLGLVHDCKRRPSRARAVSRGAKTAETVQRIKKTRRLKANNRERNRMHNLNAALDALREVLPTFPEDAKLTKIETLRFAHNYIWALTETLRLADHCGGGGGGLPGALFSEAVLLSPGGASAALSSSGDSPSPASTWSCTNSPAPSSSVSSNSTSPYSCTLSPASPAGSDMDYWQPPPPDKHRYAPHLPIARDCI

SEQ ID NO: 41 - DNA, 1153 bp, Homo sapiens
  source 1..1153: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggcttcaccatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgagacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 43 - DNA, 1609 bp, Homo sapiens
  source 1..1609: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccgcggccgcccccttcaccatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaagggtgggcgcgccgacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 45 - DNA, 1687 bp, Homo sapiens
  source 1..1687: mol_type = genomic DNA
gggttggaccctcgtacagaagctaatacgactcactatagggaaataagagagaaaagaagagtaagaagaaatataagagccaccccgcggtggcggccgctctagaactagtggatcccccgggctgcaggaattcgataaaagcgatcgcccatcacaagtttgtacaaaaaagcaggctccgcggccgcccccttcaccatgctgacccgcctgttcagcgagcccggccttctctcggacgtgcccaagttcgccagctggggcgacggcgaagacgacgagccgaggagcgacaagggcgacgcgccgccaccgccaccgcctgcgcccgggccaggggctccggggccagcccgggcggccaagccagtccctctccgtggagaagaggggacggaggccacgttggccgaggtcaaggaggaaggcgagctggggggagaggaggaggaggaagaggaggaggaagaaggactggacgaggcggagggcgagcggcccaagaagggcgggcccaagaagcgcaagatgaccaaggcgcgcttggagcgctccaagcttcggcggcagaaggcgaacgcgcgggagcgcaaccgcatgcacgacctgaacgcagccctggacaacctgcgcaaggtggtgccctgctactccaagacgcagaagctgtccaagatcgagacgctgcgcctagccaagaactatatctgggcgctctcggagatcctgcgctccggcaagcggccagacctagtgtcctacgtgcagactctgtgcaagggtctgtcgcagcccaccaccaatctggtggccggctgtctgcagctcaactctcgcaacttcctcacggagcaaggcgccgacggtgccggccgcttccacggctcgggcggcccgttcgccatgcacccctacccgtacccgtgctcgcgcctggcgggcgcacagtgccaggcggccggcggcctgggcggcggcgcggcgcacgccctgcggacccacggctactgcgcagcctacgagacgctgtatgcggcggcaggcggtggcggcgcgagcccggactacaacagctccgagtacgagggcccgctcagccccccgctctgtctcaatggcaacttctcactcaagcaggactcctcgcccgaccacgagaaaagctaccactactctatgcactactcggcgctgcccggttcgcggcccacgggccacgggctagtcttcggctcgtcggctgtgcgcgggggcgtccactcggagaatctcttgtcttacgatatgcaccttcaccacgaccggggccccatgtacgaggagctcaatgcgttttttcataactgaaagggtgggcgcgccgacccagctttcttgtacaaagtggtgatggccgctgtttaaaacttttatcaagcttatcgataccgtcgacctcgaatgctgccttctgcggggcttgccttctggccatgcccttcttctctcccttgcacctgtacctcttggtctttgaataaagcctgagtaggaagtgagggtctagaactagtgtcgacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - protein, 382 aa, Homo sapiens
MLTRLFSEPGLLSDVPKFASWGDGEDDEPRSDKGDAPPPPPPAPGPGAPGPARAAKPVPLRGEEGTEATLAEVKEEGELGGEEEEEEEEEEGLDEAEGERPKKGGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGADGAGRFHGSGGPFAMHPYPYPCSRLAGAQCQAAGGLGGGAAHALRTHGYCAAYETLYAAAGGGGASPDYNSSEYEGPLSPPLCLNGNFSLKQDSSPDHEKSYHYSMHYSALPGSRPTGHGLVFGSSAVRGGVHSENLLSYDMHLHHDRGPMYEELNAFFHN

(19) United States
(12) Patent Application Publication
(10) Pub. No.: US 2024/0384234 A1
(43) Pub. Date: Nov. 21, 2024

(54) Method For Promoting Differentiation Of Pluripotent Stem Cells By Reducing Undifferentiated State Thereof

(71) Applicant: KEIO UNIVERSITY, Tokyo (JP)

(72) Inventor: Minoru KO, Tokyo (JP)

(73) Assignee: KEIO UNIVERSITY, Tokyo (JP)

(21) Appl. No.: 18/787,081

(22) Filed: Jul. 29, 2024

Related U.S. Application Data
(63) Continuation of application No. 17/844,757, filed on Jun. 21, 2022, now Pat. No. 12,098,389.
Application No. 17/844,757 is a continuation of application No. 16/072,610, filed on Jul. 25, 2018, now Pat. No. 11,396,644.

(30) Foreign Application Priority Data
Jan. 31, 2016 (JP) 2016-016785

(51) Int. Cl. C12N 5/077; C12N 5/071; C12N 5/0793; C12N 15/09; C12N 15/113

(52) U.S. Cl. CPC C12N 5/0658; C12N 5/0619; C12N 5/067; C12N 15/09; C12N 15/113; C12N 2310/14; C12N 2506/02

(57) ABSTRACT

In related-art methods of differentiating pluripotent stem cells into a desired cell type, there has not been established a differentiation induction method using human ES/iPS cells and being highly efficient. Many attempts have been made, including a stepwise differentiation induction method based on the control of culture conditions or the addition of, for example, various cell growth factors/differentiation factors to a culture solution, but the use of complicated culture steps is a big problem. A method of inducing differentiation into a desired cell type within a short period of time and with high efficiency by use of a pluripotent stem cell that actively undergoes cell differentiation, which is obtained by reducing an undifferentiated state of the pluripotent stem cell, has been developed, and thus the present invention has been completed.

### TECHNICAL FIELD

The present invention relates to a method of reducing an undifferentiated state of a pluripotent stem cell, and more specifically, to a method of differentiating a pluripotent stem cell into a desired cell type with high efficiency and a differentiation induction kit to be used for the differentiation method.

The present application claims priority from Japanese Patent Application No. 2016-016785, which is incorporated herein by reference.

### REFERENCE TO AN ELECTRONIC SEQUENCE LISTING

The contents of the electronic sequence listing (3190-231-02-Sequence-Listing.xml; Size: 81,855 bytes; and Date of Creation: Jul. 29, 2024) is herein incorporated by reference in its entirety.

### BACKGROUND ART

### (On Induction of Differentiation of Pluripotent Stem Cells)

Regenerative medicine using cells obtained by inducing differentiation of pluripotent stem cells, such as embryonic stem cells (ES cells) or induced pluripotent stem cells (iPS cells) is a therapeutic method for which all peoples of the world have high expectations and which are desired to be realized soon. As regenerative medicine, a transplantation therapy with retinal pigment epithelial cells derived from iPS cells is fresh in our memory. However, a technology for rapidly generating mature differentiated cells suited for cell transplantation in a sufficient amount is still under development and has much room for development.

A current mainstream method of inducing differentiation of pluripotent stem cells into a desired cell type is a method involving sequentially adding cytokines/growth factors suited for respective differentiation stages to a medium to cause differentiation via an embryoid body and progenitor cells. This method has problems in, for example, that a culture period until differentiated cells of interest are obtained is long, that differentiation induction efficiency is not high, and that cells of different cell lineages are mixed with each other.

In recent years, attempts have been actively made to direct cell differentiation by forcibly expressing, in ES/iPS cells, one or a combination of a plurality of tissue-specifically expressed transcription factors. This differentiation induction method using transcription factors can directly induce ES/iPS cells into differentiated cells of interest, and hence is expected to be extremely effective means. However, even with this technique, differentiation induction efficiency for some cell types is still low, and thus, it is difficult to obtain a sufficient number of differentiated cells required for regenerative medicine.

In view of the foregoing, there has been a demand for development of a novel differentiation induction method for producing differentiated cells of interest from pluripotent stem cells more rapidly and more uniformly with higher efficiency.

### (Current Situation of Induction of Differentiation of Pluripotent Stem Cells in Related Art)

Non Patent Literatures 1 to 4, which are related art, are each directed to a system for facilitating induction of differentiation of ES/iPS cells. For an example, there was a disclosure that ES/iPS cells were induced into skeletal muscle differentiation.

However, those differentiation induction methods are clearly different from a differentiation induction method of the present invention.

In Non Patent Literature 5, there was a disclosure that “in research using mouse ES cells, when expression of a transcription factor Oct3/4 is repressed, trophectoderm can be derived from the ES cells.” In addition, in Patent Literature 1, there was a disclosure that “differentiation of neural stem cells into neurons, glial cells, and the like can be regulated by controlling an expression amount of an Oct-3/4 protein in the neural stem cells.”

However, there was no disclosure or suggestion of a method of efficiently inducing differentiation into a desired cell type by introducing a transcription factor required for induction of differentiation into the desired cell type into a pluripotent stem cell.

### CITATION LIST

### Patent Literature

[PTL 1] JP 2004-236607 A

### Non Patent Literature

[NPL 1] Nature medicine 13:642-648.
[NPL 2] Cell stem cell 10:610-619.
[NPL 3] Mol Ther. November; 20 (11): 2153-67.
[NPL 4] PLOS One. 2013 Apr. 23; 8 (4): e61540.
[NPL 5] Nat. Genet. 24 (4): 372-6.

### SUMMARY OF INVENTION

### Technical Problem

In related-art methods of differentiating pluripotent stem cells into a desired cell type, there has not been established a differentiation induction method using human ES/iPS cells and being stable and highly efficient. Many attempts have been made, including a stepwise differentiation induction method based on the control of culture conditions or the addition of, for example, various cell growth factors/differentiation factors to a culture solution, but the use of complicated culture steps is a big problem. In addition, there are also big problems in, for example, that the speed of cell differentiation is low, and hence long-period culture is required, and that the differentiation efficiency is low, and hence it is difficult to obtain a sufficient number of required cells.

### Solution to Problem

The inventors of the present invention have presumed that the above-mentioned problems are partly due to the fact that pluripotent stem cells have a property of maintaining the undifferentiated state of the cells by various mechanisms. In view of this, the inventors have developed a method of inducing differentiation into a desired cell type within a short period of time and with high efficiency by reducing undifferentiated state maintenance of a pluripotent stem cell to generate a pluripotent stem cell that actively proceeds to a differentiated cell type.

Further, the inventors have developed a method of more potently inducing differentiation into a desired cell type within a short period of time and with high efficiency by not only reducing undifferentiated state maintenance of a pluripotent stem cell but also reducing differentiation resistance thereof.

Thus, the present invention has been completed.

That is, the present invention includes the following.

1. A differentiation induction kit for differentiating a pluripotent stem cell into a desired cell type, including at least any one of the following items (1) to (8):
(1) a pluripotent stem cell and a gene for a compound having an action of substantially removing or reducing an expression amount of a POU5F1 protein;
(2) a pluripotent stem cell in which an expression amount of a POU5F1 protein has been substantially removed or reduced;
(3) a pluripotent stem cell in which a gene for a compound having an action of substantially removing or reducing an expression amount of a POU5F1 protein is forcibly expressed;
(4) a gene construct carrying a gene for a compound having an action of substantially removing or reducing an expression amount of a POU5F1 protein, and a pluripotent stem cell;
(5) a pluripotent stem cell having a gene construct carrying a gene for a compound having an action of substantially removing or reducing an expression amount of a POU5F1 protein inserted into a genome thereof;
(6) a pluripotent stem cell in which a POU5F1 gene has been disrupted;
(7) a pluripotent stem cell in which an expression amount of a POU5F1 protein has been substantially removed or reduced and which has a histone in which H3K27me3 modification has been substantially removed or reduced; and
(8) a pluripotent stem cell, and a gene for a compound having an action of substantially removing or reducing an expression amount of a POU5F1 protein and a demethylase gene.

2. A differentiation induction kit according to the above-mentioned item 1, wherein the gene for the compound having an action of substantially removing or reducing an expression amount of a POU5F1 protein includes a gene expressing siRNA of POU5F1, a gene expressing shRNA of POU5F1, a gene expressing an antisense strand of POU5F1, and/or a gene for an antibody against POU5F1.

3. A differentiation induction kit according to the above-mentioned item 1 or 2, further including a transcription factor required for induction of differentiation into the desired cell type.

4. A differentiation induction kit according to the above-mentioned item 3, wherein a transcription factor gene for the transcription factor is carried on a Sendai virus vector.

5. A differentiation induction kit for differentiating a pluripotent stem cell into a skeletal muscle cell, including at least any one of the following items (1) to (8):
(1) a pluripotent stem cell in which an expression amount of a POU5F1 protein has been substantially removed or reduced, and a transcription factor MYOD1;
(2) a gene for a compound having an action of substantially removing or reducing an expression amount of a POU5F1 protein, a transcription factor MYOD1, and a pluripotent stem cell;
(3) a pluripotent stem cell in which a gene for a compound having an action of substantially removing or reducing an expression amount of a POU5F1 protein is forcibly expressed, and a transcription factor MYOD1;
(4) a gene construct carrying a gene for a compound having an action of substantially removing or reducing an expression amount of a POU5F1 protein, a transcription factor MYOD1, and a pluripotent stem cell;
(5) a pluripotent stem cell having a gene construct carrying a gene for a compound having an action of substantially removing or reducing an expression amount of a POU5F1 protein inserted into a genome thereof, and a transcription factor MYOD1;
(6) a pluripotent stem cell in which a POU5F1 gene has been disrupted, and a transcription factor MYOD1;
(7) a transcription factor MYOD1, and a pluripotent stem cell in which an expression amount of a POU5F1 protein has been substantially removed or reduced and which has a histone in which H3K27me3 modification has been substantially removed or reduced; and
(8) a pluripotent stem cell, and a transcription factor MYOD1, a gene for a compound having an action of substantially removing or reducing an expression amount of a POU5F1 protein, and a demethylase gene.

6. A differentiation induction kit for differentiating a pluripotent stem cell into a neuron, including at least any one of the following items (1) to (8):
(1) a pluripotent stem cell in which an expression amount of a POU5F1 protein has been substantially removed or reduced, and transcription factors NEUROG1, NEUROG2, NEUROG3, NEUROD1, and NEUROD2;
(2) a gene for a compound having an action of substantially removing or reducing an expression amount of a POU5F1 protein, transcription factors NEUROG1, NEUROG2, NEUROG3, NEUROD1, and NEUROD2, and a pluripotent stem cell;
(3) a pluripotent stem cell in which a gene for a compound having an action of substantially removing or reducing an expression amount of a POU5F1 protein is forcibly expressed, and transcription factors NEUROG1, NEUROG2, NEUROG3, NEUROD1, and NEUROD2;
(4) a gene construct carrying a gene for a compound having an action of substantially removing or reducing an expression amount of a POU5F1 protein, transcription factors NEUROG1, NEUROG2, NEUROG3, NEUROD1, and NEUROD2, and a pluripotent stem cell;
(5) a pluripotent stem cell having a gene construct carrying a gene for a compound having an action of substantially removing or reducing an expression amount of a POU5F1 protein inserted into a genome thereof, and transcription factors NEUROG1, NEUROG2, NEUROG3, NEUROD1, and NEUROD2;
(6) a pluripotent stem cell in which a POU5F1 gene has been disrupted, and transcription factors NEUROG1, NEUROG2, NEUROG3, NEUROD1, and NEUROD2;
(7) transcription factors NEUROG1, NEUROG2, NEUROG3, NEUROD1, and NEUROD2, and a pluripotent stem cell in which an expression amount of a POU5F1 protein has been substantially removed or reduced and which has a histone in which H3K27me3 modification has been substantially removed or reduced; and
(8) a pluripotent stem cell, and transcription factors NEUROG1, NEUROG2, NEUROG3, NEUROD1, and NEUROD2, a gene for a compound having an action of substantially removing or reducing an expression amount of a POU5F1 protein, and a demethylase gene.

7. A differentiation induction kit for differentiating a pluripotent stem cell into a hepatocyte, including at least any one of the following items (1) to (8):
(1) a pluripotent stem cell in which an expression amount of a POU5F1 protein has been substantially removed or reduced, and a transcription factor HNF1A;
(2) a gene for a compound having an action of substantially removing or reducing an expression amount of a POU5F1 protein, a transcription factor HNF1A, and a pluripotent stem cell;
(3) a pluripotent stem cell in which a gene for a compound having an action of substantially removing or reducing an expression amount of a POU5F1 protein is forcibly expressed, and a transcription factor HNF1A;
(4) a gene construct carrying a gene for a compound having an action of substantially removing or reducing an expression amount of a POU5F1 protein, a transcription factor HNF1A, and a pluripotent stem cell;
(5) a pluripotent stem cell having a gene construct carrying a gene for a compound having an action of substantially removing or reducing an expression amount of a POU5F1 protein inserted into a genome thereof, and a transcription factor HNF1A;
(6) a pluripotent stem cell in which a POU5F1 gene has been disrupted, and a transcription factor HNF1A;
(7) a transcription factor HNF1A, and a pluripotent stem cell in which an expression amount of a POU5F1 protein has been substantially removed or reduced and which has a histone in which H3K27me3 modification has been substantially removed or reduced; and
(8) a pluripotent stem cell, a transcription factor HNF1A, a demethylase gene, and a gene for a compound having an action of substantially removing or reducing an expression amount of a POU5F1 protein.

8. A differentiation induction kit according to the above-mentioned item 3 or 4, wherein the differentiation induction kit is as described in any one of the following items (1) to (6):
(1) the transcription factor required for induction of differentiation into the desired cell type includes MYOD1, and the desired cell type includes a skeletal muscle cell;
(2) the transcription factor required for induction of differentiation into the desired cell type includes HNF1A, and the desired cell type includes a hepatocyte;
(3) the transcription factor required for induction of differentiation into the desired cell type includes SOX9, and the desired cell type includes a chondrocyte;
(4) the transcription factor required for induction of differentiation into the desired cell type includes RUNX2, and the desired cell type includes a bone cell;
(5) the transcription factor required for induction of differentiation into the desired cell type includes SPI1, and the desired cell type includes a hematopoietic cell; and
(6) the transcription factor required for induction of differentiation into the desired cell type includes ASCL1, and the desired cell type includes a neuron.

9. A method of differentiating a pluripotent stem cell into a desired cell type, including any one of the following steps (1) to (9):
(1) a step of adding a gene for a compound having an action of substantially removing or reducing an expression amount of a POU5F1 protein, and a transcription factor required for induction of differentiation into the desired cell type to a pluripotent stem cell;
(2) a step of inserting a gene construct carrying a gene for a compound having an action of substantially removing or reducing an expression amount of a POU5F1 protein, and a transcription factor gene required for induction of differentiation into the desired cell type into a genome of a pluripotent stem cell;
(3) a step of inserting a gene construct carrying a gene for a compound having an action of substantially removing or reducing an expression amount of a POU5F1 protein, and a gene construct carrying a transcription factor required for induction of differentiation into the desired cell type into a genome of a pluripotent stem cell;
(4) a step of adding a transcription factor required for induction of differentiation into the desired cell type to a pluripotent stem cell, in which an expression amount of a POU5F1 protein has been substantially removed or reduced;
(5) a step of adding a transcription factor required for induction of differentiation into the desired cell type to a pluripotent stem cell, in which a compound having an action of substantially removing or reducing an expression amount of a POU5F1 protein is forcibly expressed;
(6) a step of adding a compound having an action of substantially removing or reducing an expression amount of a POU5F1 protein, and a transcription factor required for differentiation into the desired cell type to a pluripotent stem cell;
(7) a step of adding a transcription factor required for induction of differentiation into the desired cell type to a pluripotent stem cell, in which a POU5F1 gene has been disrupted;
(8) a step of adding a gene for a compound having an action of substantially removing or reducing an expression amount of a POU5F1 protein, a demethylase gene, and a transcription factor required for induction of differentiation into the desired cell type to a pluripotent stem cell; and
(9) a step of adding a transcription factor required for induction of differentiation into the desired cell type to a pluripotent stem cell in which an expression amount of a POU5F1 protein has been substantially removed or reduced and which has a histone in which H3K27me3 modification has been substantially removed or reduced.

10. A differentiation method according to the above-mentioned item 9, wherein the gene for the compound having an action of substantially removing or reducing an expression amount of a POU5F1 protein includes a gene expressing siRNA of POU5F1, a gene expressing shRNA of POU5F1, a gene expressing an antisense strand of POU5F1, and/or a gene for an antibody against POU5F1.

11. A differentiation method according to the above-mentioned item 9 or 10, wherein a gene for the transcription factor is carried on a Sendai virus vector.

12. A method of differentiating a pluripotent stem cell into a skeletal muscle cell, including any one of the following steps (1) to (9):
(1) a step of adding a gene for a compound having an action of substantially removing or reducing an expression amount of a POU5F1 protein and a transcription factor MYOD1 to a pluripotent stem cell;
(2) a step of inserting a gene construct carrying a gene for a compound having an action of substantially removing or reducing an expression amount of a POU5F1 protein and a gene for a transcription factor MYOD1 into a genome of a pluripotent stem cell;
(3) a step of inserting a gene construct carrying a gene for a compound having an action of substantially removing or reducing an expression amount of a POU5F1 protein and a gene construct carrying a transcription factor MYOD1 into a genome of a pluripotent stem cell;
(4) a step of adding a transcription factor MYOD1 to a pluripotent stem cell in which an expression amount of a POU5F1 protein has been substantially removed or reduced;
(5) a step of adding a transcription factor MYOD1 to a pluripotent stem cell in which a compound having an action of substantially removing or reducing an expression amount of a POU5F1 protein is forcibly expressed;
(6) a step of adding a compound having an action of substantially removing or reducing an expression amount of a POU5F1 protein and a transcription factor MYOD1 to a pluripotent stem cell;
(7) a step of adding a transcription factor MYOD1 to a pluripotent stem cell in which a POU5F1 gene has been disrupted;
(8) a step of adding a gene for a compound having an action of substantially removing or reducing an expression amount of a POU5F1 protein, a demethylase gene, and a transcription factor MYOD1 to a pluripotent stem cell; and
(9) a step of adding a transcription factor MYOD1 to a pluripotent stem cell in which an expression amount of a POU5F1 protein has been substantially removed or reduced and which has a histone in which H3K27me3 modification has been substantially removed or reduced.

13. A method of differentiating a pluripotent stem cell into a neuron, including any one of the following steps (1) to (9):
(1) a step of adding a gene for a compound having an action of substantially removing or reducing an expression amount of a POU5F1 protein and transcription factors NEUROG1, NEUROG2, NEUROG3, NEUROD1, and/or NEUROD2 to a pluripotent stem cell;
(2) a step of inserting a gene construct carrying a gene for a compound having an action of substantially removing or reducing an expression amount of a POU5F1 protein and genes for transcription factors NEUROG1, NEUROG2, NEUROG3, NEUROD1, and/or NEUROD2 into a genome of a pluripotent stem cell;
(3) a step of inserting a gene construct carrying a gene for a compound having an action of substantially removing or reducing an expression amount of a POU5F1 protein and a gene construct carrying transcription factors NEUROG1, NEUROG2, NEUROG3, NEUROD1, and/or NEUROD2 into a genome of a pluripotent stem cell;
(4) a step of adding transcription factors NEUROG1, NEUROG2, NEUROG3, NEUROD1, and/or NEUROD2 to a pluripotent stem cell in which an expression amount of a POU5F1 protein has been substantially removed or reduced;
(5) a step of adding transcription factors NEUROG1, NEUROG2, NEUROG3, NEUROD1, and/or NEUROD2 to a pluripotent stem cell in which a compound having an action of substantially removing or reducing an expression amount of a POU5F1 protein is forcibly expressed;
(6) a step of adding a compound having an action of substantially removing or reducing an expression amount of a POU5F1 protein and transcription factors NEUROG1, NEUROG2, NEUROG3, NEUROD1, and/or NEUROD2 to a pluripotent stem cell;
(7) a step of adding transcription factors NEUROG1, NEUROG2, NEUROG3, NEUROD1, and/or NEUROD2 to a pluripotent stem cell in which a POU5F1 gene has been disrupted;
(8) a step of adding a gene for a compound having an action of substantially removing or reducing an expression amount of a POU5F1 protein, a demethylase gene, and transcription factors NEUROG1, NEUROG2, NEUROG3, NEUROD1, and/or NEUROD2 to a pluripotent stem cell; and
(9) a step of adding transcription factors NEUROG1, NEUROG2, NEUROG3, NEUROD1, and/or NEUROD2 to a pluripotent stem cell in which an expression amount of a POU5F1 protein has been substantially removed or reduced and which has a histone in which H3K27me3 modification has been substantially removed or reduced.

14. A method of differentiating a pluripotent stem cell into a hepatocyte, including any one of the following steps (1) to (9):
(1) a step of adding a gene for a compound having an action of substantially removing or reducing an expression amount of a POU5F1 protein and a transcription factor HNF1A to a pluripotent stem cell;
(2) a step of inserting a gene construct carrying a gene for a compound having an action of substantially removing or reducing an expression amount of a POU5F1 protein and a gene for a transcription factor HNF1A into a genome of a pluripotent stem cell;
(3) a step of inserting a gene construct carrying a gene for a compound having an action of substantially removing or reducing an expression amount of a POU5F1 protein and a gene construct carrying a transcription factor HNF1A into a genome of a pluripotent stem cell;
(4) a step of adding a transcription factor HNF1A to a pluripotent stem cell in which an expression amount of a POU5F1 protein has been substantially removed or reduced;
(5) a step of adding a transcription factor HNF1A to a pluripotent stem cell in which a compound having an action of substantially removing or reducing an expression amount of a POU5F1 protein is forcibly expressed;
(6) a step of adding a compound having an action of substantially removing or reducing an expression amount of a POU5F1 protein and a transcription factor HNF1A to a pluripotent stem cell;
(7) a step of adding a transcription factor HNF1A to a pluripotent stem cell in which a POU5F1 gene has been disrupted;
(8) a step of adding a gene for a compound having an action of substantially removing or reducing an expression amount of a POU5F1 protein, a demethylase gene, and a transcription factor HNF1A to a pluripotent stem cell; and
(9) a step of adding a transcription factor HNF1A to a pluripotent stem cell in which an expression amount of a POU5F1 protein has been substantially removed or reduced and which has a histone in which H3K27me3 modification has been substantially removed or reduced.

15. A differentiation method according to any one of the above-mentioned items 9 to 11, wherein the differentiation method is as described in any one of the following items (1) to (6):
(1) the transcription factor required for induction of differentiation into the desired cell type includes MYOD1, and the desired cell type includes a skeletal muscle cell;
(2) the transcription factor required for induction of differentiation into the desired cell type includes HNF1A, and the desired cell type includes a hepatocyte;
(3) the transcription factor required for induction of differentiation into the desired cell type includes SOX9, and the desired cell type includes a chondrocyte;
(4) the transcription factor required for induction of differentiation into the desired cell type includes RUNX2, and the desired cell type includes a bone cell;
(5) the transcription factor required for induction of differentiation into the desired cell type includes SPI1, and the desired cell type includes a hematopoietic cell; and
(6) the transcription factor required for induction of differentiation into the desired cell type includes ASCL1, and the desired cell type includes a neuron.

### Advantageous Effects of Invention

The method of differentiating a pluripotent stem cell into a desired cell type with high efficiency and differentiation induction kit for differentiating a pluripotent stem cell into a desired cell type with high efficiency of the present invention each have at least any one or more of the following effects.
(1) The period of time required for cell differentiation starting with the pluripotent stem cell is shortened and the differentiation induction efficiency is improved.
(2) As modified synthetic mRNA for a gene is used to introduce the gene into the pluripotent stem cell, the introduced gene is not incorporated into the genome of the pluripotent stem cell, and the result is that there is no risk of canceration or the like after cell differentiation induction.
(3) In the introduction of the gene into the pluripotent stem cell using the modified synthetic mRNA, the timing and number of times of the addition of the mRNA for the gene can be easily changed, and hence optimal conditions specific to each of cell types can be selected.
(4) A method of reducing undifferentiated state maintenance of a pluripotent stem cell and a method of reducing differentiation resistance thereof are combined with each other to shorten the period of time required for cell differentiation starting with the pluripotent stem cell and improve the differentiation induction efficiency in a synergistic manner.

### BRIEF DESCRIPTION OF DRAWINGS

FIG. 1 A schematic diagram of a method of inducing differentiation of a pluripotent stem cell in which an expression amount of a POU5F1 protein has been reduced of the present invention. It is illustrated that differentiation into desired cells can be induced by introducing (adding) modified synthetic RNAs for tissue-specific transcription factors to pluripotent stem cells in which the function of POU5F1, a gene responsible for an undifferentiated state, has been suppressed by an RNA interference method.

FIG. 2A A schematic diagram of a method of reducing differentiation resistance of a pluripotent stem cell to a desired cell type of the present invention. FIG. 2B When H3K27me3 modification in a human ES or iPS cell is reduced or removed, a transcription factor (TF) binds to the promoter site of a downstream gene to enhance the expression of a group of development/differentiation-related genes, resulting in differentiation. FIG. 2C A method of inducing differentiation of a human ES cell or iPS cell by introducing modified synthetic mRNA for a demethylase, and then introducing modified synthetic mRNA for the transcription factor (TF). FIG. 2D A method of inducing differentiation of a human ES cell or iPS cell by simultaneously introducing the modified synthetic mRNAs for the demethylase and the transcription factor (TF).

FIG. 3 A schematic view of a differentiation induction method using modified synthetic mRNA for a target gene.

FIG. 4 A schematic view of a differentiation step using modified synthetic mRNA for a target gene.

FIG. 5 A method of introducing a target gene into the genome of a pluripotent stem cell.

FIG. 6 The suppression of the expression of the POU5F1 protein by siRNA. Human ES cells were transfected with siRNA of POU5F1 (siPOU5F1), and it was confirmed by western blot using a specific antibody that the protein of POU5F1 was decreased (left figures) and it was confirmed by an immunostaining method using a specific antibody that the proteins of POU5F1 and NANOG were decreased (right figures). As a control, scramble siRNA (siControl) not acting on any gene was used. β-ACTIN represents a “loading control”, and DAPI represents “cell nuclei”.

FIG. 7 The morphological change of pluripotent stem cells by siPOU5F1. It is shown that human ES cells changed into flat shapes when transfected with siPOU5F1.

FIG. 8 It is shown that the co-transfection of siPOU5F1 and transcription factor mRNA activates differentiation genes. It was confirmed by a real-time PCR method that the transfection of human ES cells with modified synthetic RNAs (synRNAs) for tissue-specific transcription factors (MYOD1, HNF1A, RUNX2, SOX9, SPI1, and ASCL1) in combination with siPOU5F1 increased the expression of respective differentiation marker genes (MYOG, AFP, COL1A1, COL2A1, CD45, and NESTIN). Right bars represent cases of transfection with siPOU5F1, and left bars represent cases of transfection with siControl. mCherry or Emerald was used as synRNA serving as a negative control.

FIG. 9 Skeletal muscle differentiation by siPOU5F1 and MYOD1-synRNA was confirmed by an immunostaining method using an MyHC antibody. It was confirmed that, although MYOD1 alone (siControl+MYOD1) hardly caused muscle differentiation, its combination with siPOU5F1 dramatically induced muscle differentiation. MyHC represents a “terminal differentiation marker”, and DAPI represents “cell nuclei”.

FIG. 10 Skeletal muscle differentiation in more than one ES cell lines and iPS cell lines by siPOU5F1 and MYOD1-synRNA was confirmed by an immunostaining method using an MyHC antibody. The co-expression of siPOU5F1 and MYOD1-synRNA was able to induce skeletal muscle differentiation rapidly and with high efficiency also in an H9 line serving as an ES cell line, and four kinds of iPS cell lines (201B7, 409B2, RIKEN-1A, and tkDA3-4).

FIG. 11 Hepatocyte differentiation by siPOU5F1 and HNF1A-synRNA is shown by an immunostaining method using an albumin antibody and an AFP antibody. DAPI represents “cell nuclei”.

FIG. 12 It was confirmed by a real-time PCR method that differentiation induction by siPOU5F1 was further facilitated by combining forced expression of a demethylase therewith. It was confirmed that the expression of a differentiation marker gene (MYOG, AFP, COL2A, or CD45) activated by siPOU5F1 and a tissue-specific transcription factor (MYOD1, HNF1A, SOX9, or SPI1) was further upregulated by forced expression of the demethylase in a synergistic manner.

### DESCRIPTION OF EMBODIMENTS

A method of reducing an undifferentiated state of a pluripotent stem cell of the present invention (hereinafter sometimes referred to as “method of the present invention”) is described below, though the method is not particularly limited as long as the method is a method capable of reducing undifferentiated state maintenance of a pluripotent stem cell, and further, is a method capable of not only reducing undifferentiated state maintenance of a pluripotent stem cell but also reducing differentiation resistance thereof as required.

### (Pluripotent Stem Cell)

The pluripotent stem cell to be used in the method of the present invention is not particularly limited, but is preferably derived from a mammal, particularly preferably derived from a human. The pluripotent stem cell is, for example, a human ES cell, a human iPS cell, or any combination thereof, is not particularly limited, and encompasses tissue stem cells derived from tissues and organs, dermal fibroblasts, and all kinds of cells derived from tissues or organs.

### (Reducing Undifferentiated State Maintenance of Pluripotent Stem Cell)

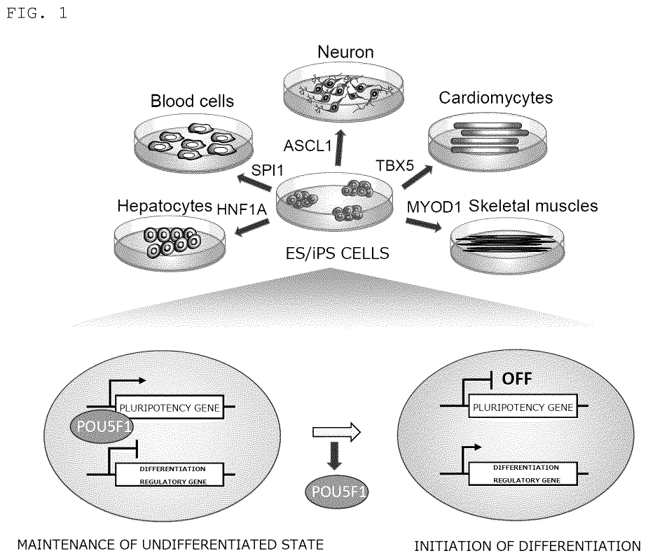

In pluripotent stem cells, the expression of a transcription factor POU5F1 (SEQ ID NOS: 1 and 2: POU domain, 5, transcription class factor 1 isoform 1: http://www.ncbi.nlm.nih.gov/protein/NP_002692, other names: OCT3, OCT4, OTF3, OTF4, OTF-3, Oct-3, Oct-4, MGC22487) is essential to the undifferentiated state maintenance of the pluripotent stem cells. POU5F1 is specifically expressed in pluripotent cells, such as reproductive cells and a preimplantation early embryo. In Examples of the present invention, it has been confirmed that differentiation into a desired cell type can be efficiently induced by introducing a transcription factor required for induction of differentiation into the desired cell type into a pluripotent stem cell in which an expression amount of a POU5F1 protein has been reduced (see FIG. 1). That is, the “reducing undifferentiated state maintenance of a pluripotent stem cell” of the present invention means substantially removing or reducing an expression amount of a POU5F1 protein in the pluripotent stem cell. The substantially removing or reducing an expression amount of a POU5F1 protein encompasses inhibiting the process of any one of the transcription and translation stages of POU5F1 and/or inhibiting the activity of the translated POU5F1 protein, and is not particularly limited.

In addition, a state in which the expression amount of the POU5F1 protein in the pluripotent stem cell has been substantially removed or reduced may be confirmed by a comparison to the degree of the expression amount of the POU5F1 protein (or expression amount of the POU5F1 gene) in a pluripotent stem cell that has not been subjected to the removing or the reducing. For example, the state (degree) in which the expression amount of the POU5F1 protein in the pluripotent stem cell has been substantially removed or reduced is from 95 to 1, from 90 to 2, from 85 to 3, from 80 to 4, from 75 to 5, from 70 to 6, from 65 to 7, from 60 to 8, from 50 to 10, from 40 to 15, from 30 to 20, or about 25 when compared to the expression amount of the POU5F1 protein in the pluripotent stem cell that has not been removed or reduced, which is defined as 100. The degree of the expression amount of the POU5F1 protein in the pluripotent stem cell may be easily measured by using a commercially available anti-POU5F1 antibody, and the gene expression amount of POU5F1 may be measured by a method known per se.

### (Reducing Differentiation Resistance of Pluripotent Stem Cell to Desired Cell Type)

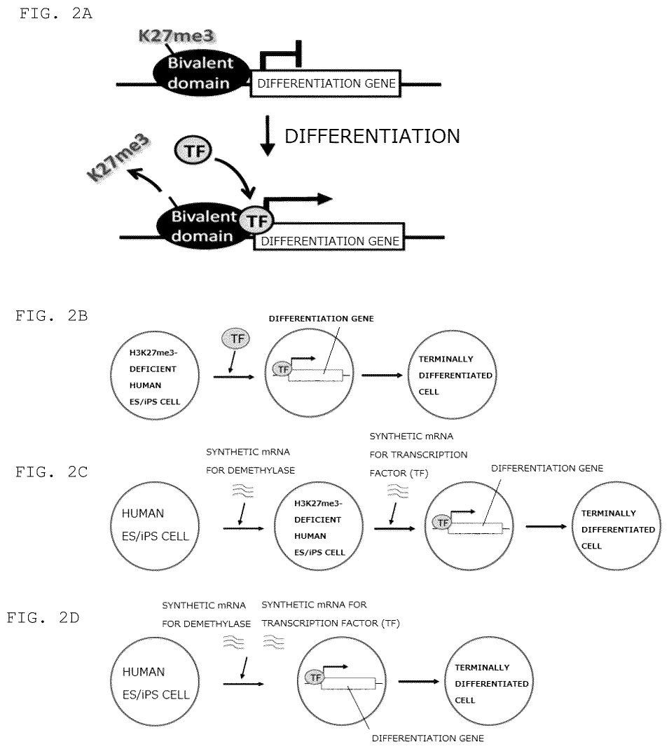

In pluripotent stem cells, a special chromatin structure called a “bivalent domain” is formed in each promoter region of a group of genes involved in differentiation, and under a stemness-maintaining state, the group of genes involved in development/differentiation are in a standby state so as not to be easily expressed. The inventors of the present invention have confirmed that “as a methyl group modification of a histone called H3K27me3 is removed or reduced in the “bivalent domain”, the expression of differentiation genes required for induction of differentiation into the desired cell type is rapidly and efficiently facilitated” (see FIGS. 2A-2D).

Namely, the “reducing differentiation resistance of a pluripotent stem cell to a desired cell type” of the present invention means that the H3K27me3 modification of the pluripotent stem cell is substantially removed or reduced.

In addition, a state in which the H3K27me3 modification of the pluripotent stem cell has been substantially removed or reduced may be confirmed by a comparison to the degree of the H3K27me3 modification of a pluripotent stem cell that has not been subjected to the removing or the reducing. For example, the state (degree) in which the H3K27me3 modification of the pluripotent stem cell has been substantially removed or reduced is from 95 to 1, from 90 to 2, from 85 to 3, from 80 to 4, from 75 to 5, from 70 to 6, from 65 to 7, from 60 to 8, from 50 to 10, from 40 to 20, or about 30 when compared to the degree of the H3K27me3 modification of the pluripotent stem cell that has not been removed or reduced, which is defined as 100. The degree of the H3K27me3 modification of the pluripotent stem cell may be easily measured by using a commercially available anti-Histone H3K27me3 antibody, and the gene expression amount of H3K27me3 may be measured by a method known per se.

(Method of Inducing Differentiation of Pluripotent Stem Cell into Desired Cell Type with High Efficiency of the Present Invention)

As described above, the method of the present invention is not particularly limited as long as the method is a method of reducing undifferentiated state maintenance of a pluripotent stem cell, and further, is a method capable of reducing differentiation resistance of a pluripotent stem cell to the desired cell type as required, and may be exemplified by the following.

(Use of Modified Synthetic mRNA for Target Gene)

The method of the present invention includes adding (introducing, transfecting), to a pluripotent stem cell, a gene for a compound having an action of substantially removing or reducing an expression amount of a POU5F1 protein (gene expressing small interfering RNA (siRNA) against POU5F1, gene expressing shRNA against POU5F1, gene expressing an antisense strand of POU5F1, or antibody gene), and further, a gene for a transcription factor required for induction of differentiation into the desired cell type.

Similarly, the method of the present invention includes adding (introducing, transfecting), to a pluripotent stem cell, a gene for a compound having an action of substantially removing or reducing H3K27me3 modification as required, and further, a gene for a transcription factor required for induction of differentiation of the pluripotent stem cell into the desired cell type.

The term “gene” as used herein encompasses not only double-stranded nucleic acids, but also their respective constituent single strands, such as plus strands (or sense strands) or complementary strands (or antisense strands), linear nucleic acids, and circular nucleic acids, and encompasses DNA, RNA, mRNA, cDNA, and the like, unless otherwise stated.

In addition, the term “target gene” is meant to encompass both of: the gene for the compound having an action of substantially removing or reducing an expression amount of a POU5F1 protein and/or the gene for the compound having an action of substantially removing or reducing H3K27me3 modification; and the transcription factor required for induction of differentiation into the desired cell type.

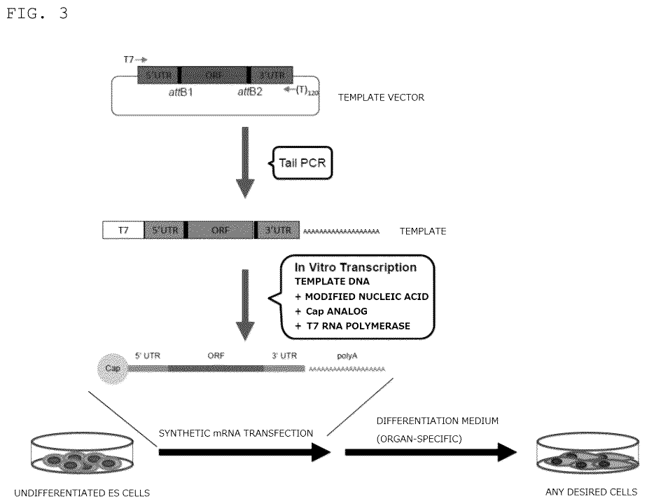

In a step of the method of the present invention, a method known per se may be used without any particular limitation as a method of adding (introducing), to the pluripotent stem cell, the gene for the compound having an action of substantially removing or reducing an expression amount of a POU5F1 protein (or the gene for the compound having an action of substantially removing or reducing H3K27me3 modification) and/or the transcription factor required for induction of differentiation into the desired cell type. There is preferably used a method of inducing differentiation by efficiently introducing synthetic mRNA for a transcription factor into human pluripotent stem cells through use of a gene expression method involving using synthetic mRNA developed by Warren, Rossi, et al. (reference: Cell Stem Cell 7:618-630, 2010), which is a footprint-free forced gene expression method causing no gene incorporation into a host genome (see FIG. 3).

The timing at which the gene for the compound having an action of substantially removing or reducing an expression amount of a POU5F1 protein (or the gene for the compound having an action of substantially removing or reducing H3K27me3 modification) and the transcription factor required for induction of differentiation into the desired cell type are added to the pluripotent stem cell is not particularly limited, but it is preferred that the gene for the compound having an action of substantially removing or reducing an expression amount of a POU5F1 protein (or the gene for the compound having an action of substantially removing or reducing H3K27me3 modification) be added to the pluripotent stem cell before the addition of the transcription factor required for differentiation induction.

Further, with regard to the addition timing of each gene (mRNA), the addition may be performed, for example, one or more times, preferably two to five times, two to four times, two or three times, or two times every 12 hours to 64 hours, but the addition timing is not particularly limited thereto. A more specific method may be exemplified by the following.

(Synthesis of Modified mRNA Encoding Amino Acid Sequence of Transcription Factor)

Modified mRNA is synthesized with a referred method described in the literature “Warren et al., Cell Stem Cell, 2010 Nov. 5; 7 (5): 618-30.” More specifically, mRNA is synthesized by in vitro transcription using a mixture of dNTPs {(dNTPs: 3-0-Me-m⁷G(5′)ppp(5′)G ARCA cap analog, 5-methylcytidine triphosphate, and pseudouridine triphosphate)} obtained by modifying template DNA encoding the amino acid sequence of the transcription factor required for induction of differentiation into the desired cell type.

### (Generation of Sendai Virus Vector Encoding Amino Acid Sequence of Transcription Factor)

In order to express a mammalian (in particular, human) transcription factor, a Sendai virus vector capable of expressing a human transcription factor is preferably used. In particular, a mutant of a Sendai virus vector, such as an F protein-deficient mutant, has no infectivity, and is easy to handle (see Inoue et al., J Virol. 77:23238-3246, 2003).

(Method of Inducing Differentiation of Pluripotent Stem Cell into Desired Cell Type with High Efficiency)

A single transcription factor or a cocktail of two or more transcription factors required for induction of differentiation into the desired cell type is prepared. The form of the transcription factors is not particularly limited, and may be any of synthetic mRNAs, a Sendai virus vector having incorporated therein a transcription factor (or a plurality of transcription factors), and nanoparticle capsules containing synthetic mRNAs.

A method of introducing the single transcription factor or cocktail of two or more transcription factors described above into cells is not particularly limited, and transfection with Lipofectamine, viral infection, or the like is utilized. A schematic view of one example of the differentiation induction step that may be utilized in the method of the present invention is illustrated in FIG. 4.

### (Use of Expression Vector)

In a step of the method of the present invention, an expression vector known per se having introduced therein the gene for the compound having an action of substantially removing or reducing an expression amount of a POU5F1 protein (or the gene for the compound having an action of substantially removing or reducing H3K27me3 modification) and/or the transcription factor required for induction of differentiation into the desired cell type may be used. Examples of the expression vector to be used in the present invention may include, but not particularly limited to, an animal cell expression plasmid vector, a Sendai virus vector and others.

A method of introducing the synthetic mRNA and the expression vector into the pluripotent stem cell is not particularly limited, but examples thereof may include a lipofection method, a liposome method, an electroporation method, a calcium phosphate coprecipitation method, a diethylaminoethyl (DEAE)-dextran method, a microinjection method, a gene gun method and others. A particularly preferred example is a lipofection method.

Another method may involve using an expression vector for the gene for the compound having an action of substantially removing or reducing an expression amount of a POU5F1 protein (or the gene for the compound having an action of substantially removing or reducing H3K27me3 modification), and using synthetic mRNA for the transcription factor required for induction of differentiation into the desired cell type, or may adopt the opposite pattern.

### (Compound Having Action of Substantially Removing or Reducing Expression Amount of POU5F1 Protein)

The compound having an action of substantially removing or reducing an expression amount of a POU5F1 protein of the present invention is not particularly limited, but is, for example, siRNA against POU5F1, shRNA against POU5F1, an antisense strand of POU5F1, an antibody that specifically binds to the POU5F1 protein, or an inhibitor.

In addition, not only by using those compounds alone, but also by using a plurality of kinds of compounds and/or a low-molecular-weight compound in combination, it is possible to efficiently “reduce an undifferentiated state of a pluripotent stem cell (substantially remove or reduce an expression amount of a POU5F1 protein in a pluripotent stem cell).”

### (Compound Having Action of Substantially Removing or Reducing H3K27Me3 Modification)

The compound having an action of substantially removing or reducing H3K27me3 modification of the present invention is not particularly limited, but is, for example, a demethylase (in particular, a demethylase having an action of removing a methyl group of H3K27me3), an antibody that specifically binds to H3K27me3, an antibody for Polycomb-group proteins (PcG proteins) having an H3K27me3 modification action, siRNA (in particular, cationic siRNA), or an inhibitor. The cationic siRNA does not require a reagent for transfection.

Examples of the low-molecular-weight compound may include, but not particularly limited to, histone deaceylase (HDAC) inhibitors, such as valproic acid.

Examples of the demethylase include AOF (LSD1), AOF1 (LSD2), FBXL11 (JHDM1A), Fbx110 (JHDM1B), FBXL19 (JHDM1C), KIAA1718 (JHDM1D), PHF2 (JHDM1E), PHF8 (JHDMIF), JMJD1A (JHDM2A), JMJD1B (JHDM2B), JMJD1C (JHDM2C), JMJD2A (JHDM3A), JMJD2B (JHDM3B), JMJD2C (JHDM3C), JMJD2D (JHDM3D), RBP2 (JARID1A), PLU1 (JARID1B), SMCX (JARID1C), SMCY (JARID1D), Jumonji (JARID2), UTX (UTX), UTY (UTY), JMJD3 (JMJD3), JMJD4 (JMJD4), JMJD5 (JMJD5), JMJD6 (JMJD6), JMJD7 (JMJD7), and JMJD8 (JMJD8). Of those, JMJD3, UTX, or the like is preferred as a demethylase having an action of removing a methyl group of H3K27me3.

In addition, the demethylase of the present invention also includes the following:
(1) a protected derivative, sugar chain-modified derivative, acylated derivative, or acetylated derivative of any one of the demethylases described above;
(2) an enzyme that has 90% (or 92%, 94%, 96%, 98%, or 99%) or more homology to any one of the demethylases described above and has a substantially equivalent action of substantially removing or reducing H3K27me3 modification to that of the demethylase; and
(3) an enzyme that has 100 to 10, 50 to 30, 40 to 20, 10 to 5, or 5 to 1 amino acid substituted, deleted, inserted, and/or added in any one of the demethylases described above and has a substantially equivalent action of substantially removing or reducing H3K27me3 modification to that of the demethylase.

Further, the gene of the demethylase of the present invention includes the following:
(1) a gene encoding a polypeptide formed of the amino acid sequence of any one or more of the enzymes described above;
(2) a gene encoding a polypeptide that has 1 to 20 (or 1 to 15, 1 to 10, 1 to 7, 1 to 5, or 1 to 3) amino acids substituted, deleted, inserted, and/or added in the amino acid sequence of any one or more of the enzymes described above and has a substantially equivalent action of substantially removing or reducing H3K27me3 modification to that of the demethylase; and
(3) a gene encoding a polypeptide that has 90% (or 92%, 94%, 96%, 98%, or 99%) or more homology to the amino acid sequence of any one or more of the enzymes described above and has a substantially equivalent action of substantially removing or reducing H3K27me3 modification to that of the demethylase.

An enzyme having a mutation may be a naturally occurring one, or may be one obtained by introducing a mutation on the basis of a gene of natural origin. Means for introducing a mutation is known per se, and for example, a site-directed mutagenesis method, a homologous gene recombination method, a primer extension method, a polymerase chain reaction (hereinafter abbreviated as PCR), and the like may be used alone or in combination thereof as appropriate.

The method may be performed in conformity with any of methods described in the literatures (“Molecular Cloning: A Laboratory Manual, second edition” edited by Sambrook et al., 1989, Cold Spring Harbor Laboratory; and “Lab Manual: Genetic Engineering” edited by Masami Muramatsu, 1988, Maruzen), or by modifying these methods, and Ulmer's technology (Ulmer, K. M., “Science”, 1983, volume 219, p. 666-671) may also be utilized. In the case of a peptide, from the viewpoint of preventing alteration of basic properties of the peptide (e.g., physical properties, function, physiological activity, or immunological activity) in the introduction of a mutation, for example, mutual substitution between homologous amino acids (e.g., polar amino acids, non-polar amino acids, hydrophobic amino acids, hydrophilic amino acids, positively charged amino acids, negatively charged amino acids, and aromatic amino acids) is easily conceivable.

### (JMJD3)

JMJD3 is known as a demethylase for H3K27me3 of a histone (mouse NP_001017426, human NP_001073893), and even in its full length (NP_001073893, SEQ ID NO: 3), has an action of substantially removing or reducing the H3K27me3 modification of pluripotent stem cells. Surprisingly, the inventors of the present invention have confirmed that JMJD3c having the JmjC domain {SEQ ID NO: 4, catalytic domain: SEQ ID NO: 5 (amino acids 1376-1484)} has a stronger action of substantially removing or reducing H3K27me3 modification as compared to full-length JMJD3.

A preferred base sequence of JMJD3 is a base sequence set forth in SEQ ID NO: 32.

In addition, the JMJD3 of the present invention includes the following embodiments as well:
(1) a protected derivative, sugar chain-modified derivative, acylated derivative, or acetylated derivative of an amino acid sequence set forth in SEQ ID NO: 3;
(2) an amino acid sequence that has 90% (or 92%, 94%, 96%, 98%, or 99%) or more homology to the amino acid sequence set forth in SEQ ID NO: 3 and has a substantially equivalent action of substantially removing or reducing H3K27me3 modification to that of the JMJD3;
(3) an amino acid sequence that has 100 to 10, 50 to 30, 40 to 20, 10 to 5, or 5 to 1 amino acid substituted, deleted, inserted, and/or added in the amino acid sequence set forth in SEQ ID NO: 3 and has a substantially equivalent action of substantially removing or reducing H3K27me3 modification to that of the JMJD3;
(4) a protected derivative, sugar chain-modified derivative, acylated derivative, or acetylated derivative of an amino acid sequence set forth in SEQ ID NO: 4;
(5) an amino acid sequence that has 90% (or 92%, 94%, 96%, 98%, or 99%) or more homology to the amino acid sequence set forth in SEQ ID NO: 4 and has a substantially equivalent action of substantially removing or reducing H3K27me3 modification to that of the JMJD3c;
(6) an amino acid sequence that has 100 to 10, 50 to 30, 40 to 20, 10 to 5, or 5 to 1 amino acid substituted, deleted, inserted, and/or added in the amino acid sequence set forth in SEQ ID NO: 4 and has a substantially equivalent action of substantially removing or reducing H3K27me3 modification to that of the JMJD3c; and
(7) an amino acid sequence that includes the amino acid sequence set forth in SEQ ID NO: 5 and has a substantially equivalent action of substantially removing or reducing H3K27me3 modification to the JMJD3c.

It is appropriate that the “sequence homology” be generally 70% or more, preferably 80%, more preferably 85% or more, still more preferably 90% or more, even more preferably 95% or more, most preferably 98% or more of an entire amino acid sequence.

In addition, the JMJD3 gene of the present invention includes the following:
(1) a gene encoding a polypeptide consisted of an amino acid sequence set forth in any one of SEQ ID NOS: 3 to 5;
(2) a gene encoding a polypeptide that has 1 to 20 (or 1 to 15, 1 to 10, 1 to 7, 1 to 5, or 1 to 3) amino acids substituted, deleted, inserted, and/or added in the amino acid sequence set forth in any one of SEQ ID NOS: 3 to 5 and has a substantially equivalent action of substantially removing or reducing H3K27me3 modification to that of the amino acid sequence set forth in any one of SEQ ID NOS: 3 to 5;
(3) a gene encoding a polypeptide that has 90% (or 92%, 94%, 96%, 98%, or 99%) or more homology to the amino acid sequence set forth in any one of SEQ ID NOS: 3 to 5 and has a substantially equivalent action of substantially removing or reducing H3K27me3 modification to that of the amino acid sequence set forth in any one of SEQ ID NOS: 3 to 5;
(4) a gene consisted of a base sequence set forth in any one of SEQ ID NOS: 6 to 8 and 32;
(5) a gene encoding a polypeptide that hybridizes with a base sequence complementary to the base sequence set forth in any one of SEQ ID NOS: 6 to 8 and 32 under stringent conditions and has a substantially equivalent action of substantially removing or reducing H3K27me3 modification to that of the amino acid sequence set forth in any one of SEQ ID NOS: 3 to 5;
(6) a gene that has a sequence of 1 to 50 (or 1 to 40, 1 to 30, 1 to 20, 1 to 15, 1 to 10, 1 to 5, or 1 to 3 bases substituted, deleted, inserted, and/or added in the DNA consisted of the base sequence set forth in any one of SEQ ID NOS: 6 to 8 and 32; and
(7) a gene having 90% (or 92%, 94%, 96%, 98%, or 99%) or more homology to the gene formed of the base sequence set forth in any one of SEQ ID NOS: 6 to 8 and 32.
   (Transcription Factor Required for Highly Efficient Induction of Differentiation into Desired Cell Type)

The embodiment of the “transcription factor required for highly efficient induction of differentiation into the desired cell type” to be used in the method of the present invention is not particularly limited, but examples thereof may include, but not particularly limited to, nucleic acids, such as RNA and DNA, synthetic nucleic acids, and proteins.

In addition, in the method of the present invention, examples of the desired cell type may include a skeletal muscle, the liver (hepatocytes), neurons, chondrocytes, bone cells, and hematopoietic cells.

{Transcription Factor Required for Induction of Differentiation into Skeletal Muscle (in Particular, Cells Present in Skeletal Muscle)}

A method of inducing differentiation into a skeletal muscle is as described below.

A single transcription factor, or two or more transcription factors selected from the group of MYOD1, NRF1, SALL4, ZIC1, KLF9, ZNF281, CTCF, HES1, HOXA2, TBX5, TP73, ERG, MAB21L3, PRDM1, NFIC, CTCFL, FOXP1, HEY1, PITX2, JUNB, KLF4, ESX1, TFAP2C, FOS, TFE3, FOSL1, GRHL2, TBX2, NFIB, and IRF4 are introduced into pluripotent stem cells. In particular, MYOD1 is preferably introduced into pluripotent stem cells.

{Transcription Factor Required for Induction of Differentiation into Liver (in Particular, Cells Present in Liver, i.e., Hepatoblasts)}

A method of inducing differentiation into the liver (in particular, the liver or the fetal liver) is as described below.

Liver: A single transcription factor, or two or more transcription factors selected from HNF1A, TCF-1, SALL4, TGIF1, MAB21L3, ZIC1, EGFLAM, PITX2, HNF4A, NRF1, ZNF281, CTCFL, TP73, TFE3, DLX6, and TCF4 are introduced into human pluripotent stem cells.

Fetal liver: A single transcription factor, or two or more transcription factors selected from HNF1A, TCF-1, SIX5, HNF4A, SIN3A, ID1, and HNF1A are introduced into human pluripotent stem cells.

In particular, HNF1A is preferably introduced into pluripotent stem cells.

(Transcription Factor Required for Induction of Differentiation into Neurons)

A method of inducing differentiation into neurons is as described below.

A single transcription factor, or two or more, three or more, four or more, or five transcription factors selected from NEUROG1, NEUROG2, NEUROG3, NEUROD1, and NEUROD2 are introduced into human pluripotent stem cells.

(Method of Introducing Target Gene into Genome of Pluripotent Stem Cell)

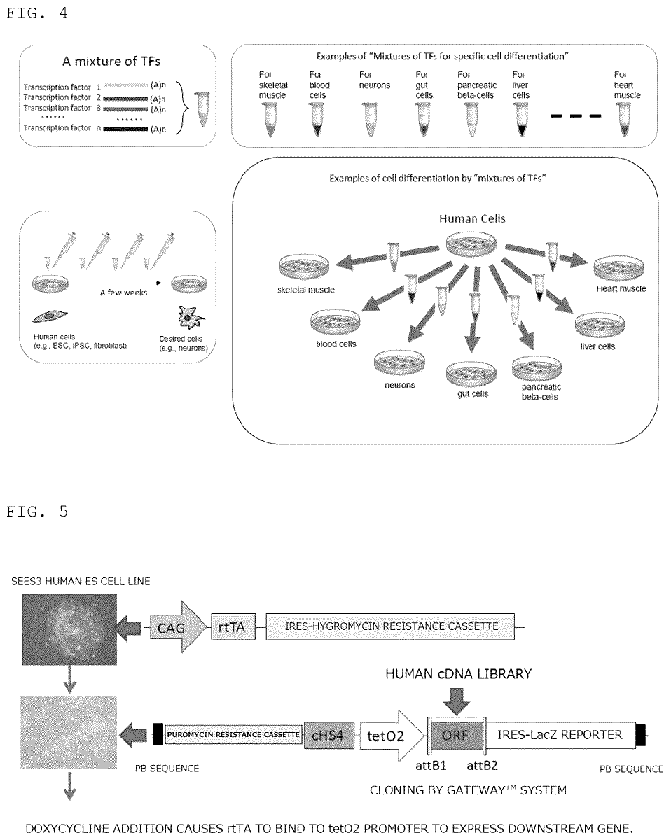

In a step of the method of the present invention, a method known per se may be used without any particular limitation as a method of introducing the gene for the compound having an action of substantially removing or reducing an expression amount of a POU5F1 protein (or the gene for the compound having an action of substantially removing or reducing H3K27me3 modification) and/or the transcription factor required for highly efficient induction of differentiation into the desired cell type into the genome of the pluripotent stem cell. There may be preferably used an expression cassette inserted between PiggyBac transposase recognition sequences (PB sequences) developed by Woltjen et al. (reference: Nature 458:766-770, 2009), which is a mechanism by which a gene to be introduced is actively incorporated into pluripotent stem cells (in particular, the genome of human ES cells). The expression cassette is a system capable of efficiently establishing a genetically modified pluripotent stem cell line by introducing a drug selection cassette (see FIG. 5).

(Method of Introducing Target Protein into Pluripotent Stem Cell)

In a step of the method of the present invention, a method known per se may be used as a method of introducing the compound having an action of substantially removing or reducing an expression amount of a POU5F1 protein (or the compound having an action of substantially removing or reducing H3K27me3 modification) (in particular, protein) and/or the transcription factor (protein) required for highly efficient induction of differentiation into the desired cell type into the genome of the pluripotent stem cell, and examples thereof may include: a method involving using a protein transfection reagent; a method involving using a fusion protein having added thereto a cell-penetrating peptide; and a microinjection method.

The “cell-penetrating peptide” of the present invention is a peptide having a property of migrating into a cell, more specifically a property of penetrating a cell membrane, still more specifically a property of penetrating a cell membrane or a nuclear membrane to penetrate into cytoplasm or a nucleus. The amino acid sequence of the peptide is not particularly limited, but examples thereof may include TAT (GRKKRRQRRRPQ: SEQ ID NO: 9), r8 {rrrrrrrr (D-form-R): SEQ ID NO: 10}, and MPG-8 (BAFLGWLGAWGTMGWSPKKKRK: SEQ ID NO: 11).

The target protein encompasses both of the compound having an action of substantially removing or reducing an expression amount of a POU5F1 protein (or the compound having an action of substantially removing or reducing H3K27me3 modification) (in particular, protein) and/or the transcription factor (protein) required for highly efficient induction of differentiation into the desired cell type.

### (Gene Knockout Method)

A gene knockout method is available as a method other than the foregoing. A “pluripotent stem cell in which a POU5F1 gene has been disrupted” may be generated by the gene knockout method. The “pluripotent stem cell in which a POU5F1 gene has been disrupted” means that normal expression of the POU5F1 gene is inhibited due to artificial modification of the sequence of a POU5F1 gene region, and as a result, the expression of POU5F1 is suppressed and a POU5F1 protein is not normally expressed.

In addition, the “whole” in “modification or deletion of part or the whole of the POU5F1 gene” refers to the protein-coding region of POU5F1 genomic DNA.

In addition, the “part” refers to a region that is part of the protein-coding region and that has a length required for inhibiting normal expression of the POU5F1 gene.

Further, the “modification” refers to modification of the base sequence of a target region in genomic DNA into another base sequence by substituting, deleting, inserting, and/or adding a single nucleotide or two or more of nucleotides.

(Differentiation Induction Kit for Inducing Differentiation of Pluripotent Stem Cell into Desired Cell Type with High Efficiency)

A differentiation induction kit for inducing differentiation of a pluripotent stem cell into a desired cell type with high efficiency of the present invention (hereinafter sometimes referred to as “kit of the present invention”) includes any one or more of the following modes.

(1) Pluripotent Stem Cell in which Expression Amount of POU5F1 Protein has been Substantially Removed or Reduced and/or H3K27Me3 Modification has been Substantially Removed or Reduced

A pluripotent stem cell in which an expression amount of a POU5F1 protein has been substantially removed or reduced and/or H3K27me3 modification has been substantially removed or reduced can be easily generated by the method of the present invention described above.

A implementer can easily induce differentiation into the desired cell type by introducing the transcription factor required for induction of differentiation into the desired cell type as described above into the pluripotent stem cell.

In addition, such pluripotent stem cell encompasses a pluripotent stem cell having a gene construct inducible with doxycycline or the like inserted into the genome thereof so that a gene for a compound having an action of substantially removing or reducing an expression amount of a POU5F1 protein, a demethylase, or the like can be transiently forcibly expressed therein.

(2) Gene for Compound Having Action of Substantially Removing or Reducing Expression Amount of POU5F1 Protein and/or Demethylase Gene for Kit of the Present Invention

The implementer can easily generate the pluripotent stem cell in which an expression amount of a POU5F1 protein has been substantially removed or reduced and/or H3K27me3 modification has been substantially removed or reduced by adding a gene for a compound having an action of substantially removing or reducing an expression amount of a POU5F1 protein and/or a demethylase gene for a kit to a pluripotent stem cell.

Examples of the anti-POU5F1 antibody gene for a kit may include, but not particularly limited to, commercially available antibody genes.

Examples of the demethylase gene for a kit may include, but not particularly limited to, mRNAs, DNAs, and proteins of demethylase genes (e.g., JMJD3c).

(3) Gene for Compound Having Action of Substantially Removing or Reducing Expression Amount of POU5F1 Protein and/or Demethylase Gene, and Gene Containing Transcription Factor Required for Induction of Differentiation into Desired Cell Type for Kit of the Present Invention

The implementer can easily generate the pluripotent stem cell in which an expression amount of a POU5F1 protein has been substantially removed or reduced and/or H3K27me3 modification has been substantially removed or reduced, and further, can induce differentiation thereof into the desired cell type with high efficiency by adding a gene for a compound having an action of substantially removing or reducing an expression amount of a POU5F1 protein and/or a demethylase gene, and a gene containing a transcription factor required for induction of differentiation into the desired cell type for a kit to a pluripotent stem cell.

Those genes may be present on one gene, or on separate genes. When the genes are present on separate genes, the gene for the compound having an action of substantially removing or reducing an expression amount of a POU5F1 protein (or demethylase gene) and the transcription factor required for induction of differentiation into the desired cell type may be added to the pluripotent stem cell simultaneously or at separate times.

(4) Compound Having Action of Substantially Removing or Reducing Expression Amount of POU5F1 Protein and/or Demethylase for Kit of the Present Invention

The implementer can easily generate the pluripotent stem cell in which an expression amount of a POU5F1 protein has been substantially removed or reduced and/or H3K27me3 modification has been substantially removed or reduced by adding a compound having an action of substantially removing or reducing an expression amount of a POU5F1 protein and/or a demethylase for a kit to a pluripotent stem cell.

(5) Gene Construct Carrying Gene for Compound Having Action of Substantially Removing or Reducing Expression Amount of POU5F1 Protein and/or Demethylase Gene

The user can easily generate the pluripotent stem cell in which an expression amount of a POU5F1 protein has been substantially removed or reduced and/or H3K27me3 modification has been substantially removed or reduced by introducing a gene construct carrying a gene for a compound having an action of substantially removing or reducing an expression amount of a POU5F1 protein and/or a demethylase gene into the genome of a pluripotent stem cell.

The gene construct may contain a promoter sequence, a gene expression-enhancing sequence, a marker gene, a reporter sequence, a drug resistance gene, and the like as required in addition to the gene for the compound having an action of substantially removing or reducing an expression amount of a POU5F1 protein and/or the demethylase gene.

(6) Gene Construct Carrying: Gene for Compound Having Action of Substantially Removing or Reducing Expression Amount of POU5F1 Protein and/or Demethylase Gene; and Transcription Factor Required for Induction of Differentiation into Desired Cell Type

The implementer can easily generate the pluripotent stem cell in which an expression amount of a POU5F1 protein has been substantially removed or reduced and/or H3K27me3 modification has been substantially removed or reduced, and further, can induce differentiation thereof into the desired cell type by introducing a gene construct carrying: a gene for a compound having an action of substantially removing or reducing an expression amount of a POU5F1 protein and/or a demethylase gene; and a transcription factor required for induction of differentiation into the desired cell type into the genome of a pluripotent stem cell.

Those genes may be present on one gene, or on separate genes. When the genes are present on separate genes, the gene for the compound having an action of substantially removing or reducing an expression amount of a POU5F1 protein and/or the demethylase gene, and the transcription factor required for induction of differentiation into the desired cell type may be expressed in the genome of the pluripotent stem cell simultaneously or at separate times.

The gene construct may contain a promoter sequence, a gene expression-enhancing sequence, a marker gene, a reporter sequence, a drug resistance gene, and the like as required in addition to the gene for the compound having an action of substantially removing or reducing an expression amount of a POU5F1 protein and/or the demethylase gene, and the transcription factor required for induction of differentiation into the desired cell type.

(Proteins Associated with Undifferentiated State Maintenance Other than POU5F1)

NANOG, SOX2, SOX3, KLF2, KLF4, KLF5, TBX3, ESRRB, SALLA, STAT3, ZIC3, LIN28, and TCF3 are known as proteins associated with undifferentiated state maintenance. Accordingly, it is considered that pluripotent stem cells in which expression amounts of the proteins of NANOG, SOX2, SOX3, KLF2, KLF4, KLF5, TBX3, ESRRB, SALL4, STAT3, ZIC3, LIN28, and TCF3 have been substantially removed or reduced can be differentiated into the desired cell type with high efficiency.

Further, a pluripotent stem cell in which an expression amount of any one or more of the above-mentioned proteins has been substantially removed or reduced and an expression amount of a POU5F1 protein has been substantially removed or reduced (pluripotent stem cell in which an expression amount of any one or more of the above-mentioned proteins has been substantially removed or reduced and an expression amount of a POU5F1 protein has been substantially removed or reduced, and which has a histone in which H3K27me3 modification has been substantially removed or reduced) is also included in the present invention.

In addition, a method of differentiating a pluripotent stem cell into a desired cell type including a step of adding a gene for a compound having an action of substantially removing or reducing an expression amount of any one or more of the above-mentioned proteins, a gene for a compound having an action of substantially removing or reducing an expression amount of a POU5F1 protein (further, a demethylase gene or the like), and a transcription factor required for induction of differentiation into the desired cell type to a pluripotent stem cell is also encompassed in the present invention.

A method of differentiating a pluripotent stem cell into a desired cell type of the present disclosure may be exemplified by, but not particularly limited to, a method including any one of the following steps (1) to (9):
(1) a step of adding a gene for a compound having an action of substantially removing or reducing an expression amount of a POU5F1 protein and a transcription factor required for induction of differentiation into the desired cell type to a pluripotent stem cell;
(2) a step of inserting a gene construct carrying a gene for a compound having an action of substantially removing or reducing an expression amount of a POU5F1 protein and a transcription factor gene required for induction of differentiation into the desired cell type, into a genome of a pluripotent stem cell;
(3) a step of inserting a gene construct carrying a gene for a compound having an action of substantially removing or reducing an expression amount of a POU5F1 protein and a gene construct carrying a transcription factor required for induction of differentiation into the desired cell type, into a genome of a pluripotent stem cell;
(4) a step of adding a transcription factor required for induction of differentiation into the desired cell type to a pluripotent stem cell in which an expression amount of a POU5F1 protein has been substantially removed or reduced;
(5) a step of adding a transcription factor required for induction of differentiation into the desired cell type to a pluripotent stem cell in which a compound having an action of substantially removing or reducing an expression amount of a POU5F1 protein is forcibly expressed;
(6) a step of adding a compound having an action of substantially removing or reducing an expression amount of a POU5F1 protein and a transcription factor required for differentiation into the desired cell type to a pluripotent stem cell;
(7) a step of adding a transcription factor required for induction of differentiation into the desired cell type to a pluripotent stem cell in which a POU5F1 gene has been disrupted;
(8) a step of adding a gene for a compound having an action of substantially removing or reducing an expression amount of a POU5F1 protein, a demethylase gene, and a transcription factor required for induction of differentiation into the desired cell type to a pluripotent stem cell; and
(9) a step of adding a transcription factor required for induction of differentiation into the desired cell type to a pluripotent stem cell in which an expression amount of a POU5F1 protein has been substantially removed or reduced and which has a histone in which H3K27me3 modification has been substantially removed or reduced.

The present disclosure also includes any one of the following pluripotent stem cells for differentiation into a desired cell type:
(1) a pluripotent stem cell for differentiation into a desired cell type, in which an expression amount of a POU5F1 protein has been substantially removed or reduced;
(2) a pluripotent stem cell for differentiation into a desired cell type, in which a gene for a compound having an action of substantially removing or reducing an expression amount of a POU5F1 protein is forcibly expressed;
(3) a pluripotent stem cell for differentiation into a desired cell type, which has a gene construct carrying a gene for a compound having an action of substantially removing or reducing an expression amount of a POU5F1 protein inserted into the genome thereof;
(4) a pluripotent stem cell for differentiation into a desired cell type, in which a POU5F1 gene has been disrupted; and
(5) a pluripotent stem cell for differentiation into a desired cell type, in which an expression amount of a POU5F1 protein has been substantially removed or reduced and which has a histone in which H3K27me3 modification has been substantially removed or reduced.

The present disclosure also includes a use of any one of the following pluripotent stem cells for differentiation into a desired cell type:
(1) a pluripotent stem cell for differentiation into a desired cell type, in which an expression amount of a POU5F1 protein has been substantially removed or reduced;
(2) a pluripotent stem cell for differentiation into a desired cell type, in which a gene for a compound having an action of substantially removing or reducing an expression amount of a POU5F1 protein is forcibly expressed;
(3) a pluripotent stem cell for differentiation into a desired cell type, which has a gene construct carrying a gene for a compound having an action of substantially removing or reducing an expression amount of a POU5F1 protein inserted into the genome thereof;
(4) a pluripotent stem cell for differentiation into a desired cell type, in which a POU5F1 gene has been disrupted; and
(5) a pluripotent stem cell for differentiation into a desired cell type, in which an expression amount of a POU5F1 protein has been substantially removed or reduced and which has a histone in which H3K27me3 modification has been substantially removed or reduced.

The present disclosure also includes a use of any one of the following pluripotent stem cells for differentiation into a desired cell type, in production of a differentiation induction kit for differentiating a pluripotent stem cell into a desired cell type:
(1) a pluripotent stem cell for differentiation into a desired cell type, in which an expression amount of a POU5F1 protein has been substantially removed or reduced;
(2) a pluripotent stem cell for differentiation into a desired cell type, in which a gene for a compound having an action of substantially removing or reducing an expression amount of a POU5F1 protein is forcibly expressed;
(3) a pluripotent stem cell for differentiation into a desired cell type, which has a gene construct carrying a gene for a compound having an action of substantially removing or reducing an expression amount of a POU5F1 protein inserted into the genome thereof;
(4) a pluripotent stem cell for differentiation into a desired cell type, in which a POU5F1 gene has been disrupted; and
(5) a pluripotent stem cell for differentiation into a desired cell type, in which an expression amount of a POU5F1 protein has been substantially removed or reduced and which has a histone in which H3K27me3 modification has been substantially removed or reduced.

The present invention is more embodied described below by Examples. However, the present invention is not limited to these Examples. All of these Examples have been approved by the Ethics Committee of Keio University School of Medicine.

### Example 1

### (Materials and Methods)

Examples 2 to 6 were carried out using materials and methods described below. The details are as described below.

### (Human Pluripotent Stem Cell Culture and Differentiation Induction Methods)

Human ES cell (hESC) lines SEES-3 and H9 were obtained from the National Center for Child Health and Development (National Research Institute for Child Health and Development) and the Cell Research Institute, USA, respectively. A human induced pluripotent stem cell (hiPSC) line was obtained from RIKEN or the Center for iPS Cell Research and Application, Kyoto University. hESC/iPSCs were cultured using StemFit AK-03 medium (Ajinomoto) on iMatrix-511 (Nippi)-coated plates without use of feeder cells. A ROCK (Rho-associated coiled-coil forming kinase/Rho-associated kinase) inhibitor Y-27632 was added to the medium during cell subculture in order to inhibit apoptosis induced by cell detachment during cell passaging.

For myogenic differentiation, the hESC/iPSCs were cultured in a medium of αMEM (Gibco) supplemented with 5% KSR, 1 mM sodium pyruvate, 0.1 mM non-essential amino acids, 2 mM glutamine, 0.1 mM β-mercaptoethanol, and penicillin/streptomycin (50 U/50 μg/ml) on iMatrix-511-coated plates.

For albumin secretion hepatocyte differentiation, the hESC/iPSCs were cultured in the above-mentioned medium on Matrigel (BD)-coated plates, then cultured in RPMI 1640 medium supplemented with 1 mM NaB, 100 ng/ml Activin A, 50 ng/ml Wnt3a, 1×B27, and 2 mM GlutaMAX for 1 day, subsequently cultured in DMEM medium supplemented with 1% DMSO, 0.5 mM MTG, 1% NEAA, 1 mM GlutaMAX, and 20% KSR for 5 days, and finally cultured in HCM medium (Lonza) supplemented with 20 ng/ml HGF and 20 ng/ml Oncostatin M for 7 days.

(siRNA Transfection)

siRNA against POU5F1 (sense strand: GCCCGAAAGAGAAAGCGAATT: SEQ ID NO: 12, antisense strand: UUCGCUUUCUCUUUCGGGCCT: SEQ ID NO: 13) identical to that used in the literature “Proceeding of national academy of sciences 109, 4485-4490 (2012)” was purchased from Applied Biosystems and used (product number: s10873). siRNA serving as a negative control was also purchased from Applied Biosystems and used. siRNA transfections were performed with Lipofectamine Messenger Max (Invitrogen), according to the instructions of the accompanying manual. The B18R interferon inhibitor (eBioscience) was added to the culture medium to increase the viability of the transfected cells. The medium was replaced 2 hours to 3 hours after each transfection.

(Modified mRNA Synthesis and Transfection)

The protein-coding regions (Open Reading Frames, ORFs) of a red fluorescent protein mCherry, a green fluorescent protein Emerald, and tissue-specific transcription factors {MYOD1 (SEQ ID NO: 26), HNF1A (SEQ ID NO: 27), RUNX2 (SEQ ID NO: 28), SOX9 (SEQ ID NO: 29), SPI1 (SEQ ID NO: 30), and ASCL1 (SEQ ID NO: 31)} were subcloned into a pCRII construct containing the 5′ UTR and 3′ UTR of mouse α-globin, which increased mRNA stability and translation efficiency, to prepare templates used to synthesize mRNAs.

Modified mRNAs were synthesized on the basis of the description of the literature “Cell stem cell 7, 618-630 (2010)”. Briefly speaking, a T7 promoter and a poly(A) tail were added through PCR reaction using a KAPA taq kit (Kapa Biosystems). RNAs were synthesized from PCR products using a MEGAscript T7 kit (Ambion) together with ARCA cap analog (New England Biolabs), ATP, GTP, 5-Methyl-CTP (TriLink), and pseudo-UTP (TriLink). The synthetic mRNAs were purified using a MEGAclear kit (Ambion). Synthetic mRNA transfections were performed with Lipofectamine MessengerMax (Invitrogen) according to the instructions of the accompanying manual. The B18R interferon inhibitor (eBioscience) was added to the culture medium to increase the viability of the transfected cells. The medium was replaced 2 hours to 3 hours after each transfection.

### (Antibody)

The following antibodies were used:
POU5F1 (Santa Cruz, sc-5279);
β-ACTIN (Cell Signaling, 4970S);
MyHC (R&D MAB4470);
ALBUMIN (Abcam ab10241); and
AFP (R&D MAB1368).

### (Immunostaining)

The cells were fixed in 4% PFA for 10 minutes at room temperature and permeabilized in 0.5% Triton-X-containing PBS for 10 minutes. The cells were treated a blocking in 2% BSA-containing PBS for 10 minutes, and cultured with primary antibodies in a blocking solution (1:500) for from 2 hours to 3 hours at room temperature or overnight at 4° C. The cells were washed twice in PBS, and then cultured with Alexa dye-conjugated secondary antibodies (Invitrogen) in a blocking solution (1:500) for 1 hour at room temperature. Nuclei were counterstained with DAPI (Dako). Immunofluorescence was visualized with an inverted fluorescence microscope IX73 (Olympus). Images were obtained using Olympus cellSens imaging software.

### (Immunoblotting Method)

The cells were lysed with a sample buffer (50 mM Tris-HCl, pH 6.8, 2% SDS, 6% 2-mercaptoethanol, and 500 mg/ml urea). The proteins were separated by SDS-PAGE using a 4-15% polyacrylamide gel (Biorad) and were electrically transferred to polyvinylidene fluoride membranes (Biorad). The membranes were blocked for 1 hour in 0.1% Tween-20-containing Tris-buffered saline (TBST) and 5% skimmed milk. The membranes were washed in TBST and then incubated with primary antibodies in 2% BSA-containing TBS (1:1,000 dilution) for from 2 hours to 3 hours at room temperature or overnight at 4° C. The membranes were washed and incubated with horseradish peroxidase-conjugated secondary antibodies (GE) for 1 hour at room temperature. The membranes were washed in TBST, and immunoreactivity was visualized using ECL Prime Detection Kit (GE) and detected using Luminescent Image Analyzer (LAS-4000; Fujifilm).

(qRT-PCR)

Total RNA was isolated with TRIzol reagent (Invitrogen), and cDNAs were generated with random hexamers using a ReverTra Ace kit (Toyobo). Real-time PCR was performed using a SYBR Green PCR system (Takara). The primer sequences used for RT-PCR are listed below.
MYOG primer (Forward): gccagactatccccttcctc (SEQ ID NO: 14)
MYOG primer (Reverse): gaggccgegttatgataaaa (SEQ ID NO: 15)
AFP primer (Forward): tgggacccgaactttcca (SEQ ID NO: 16)
AFP primer (Reverse): ggccacatccaggactagtttc (SEQ ID NO: 17)
COL1A1 primer (Forward): cctggatgccatcaaagtct (SEQ ID NO: 18)
COL1A1 primer (Reverse): tcttgtccttggggttcttg (SEQ ID NO: 19)
COL2A1 primer (Forward): tttcccaggtcaagatggtc (SEQ ID NO: 20)
COL2A1 primer (Reverse): cttcagcacctgtctcacca (SEQ ID NO: 21)
CD45 primer (Forward): tcctggactcccaaaatctg (SEQ ID NO: 22)
CD45 primer (Reverse): accttgaacccgaacatgag (SEQ ID NO: 23)
NESTIN primer (Forward): tggttttccagagtcttcagtga (SEQ ID NO: 24)
NESTIN primer (Reverse): gaaacagccatagagggcaaa (SEQ ID NO: 25)

### Example 2

(Pluripotent Stem Cells in which Expression Amount of POU5F1 Protein has been Reduced)

In this Example, it was confirmed whether pluripotent stem cells in which the expression amount of a POU5F1 protein had been forcibly reduced were able to be generated or not. The details are as described below.

(Confirmation of Suppression of Expression of POU5F1 by siRNA Transfection)

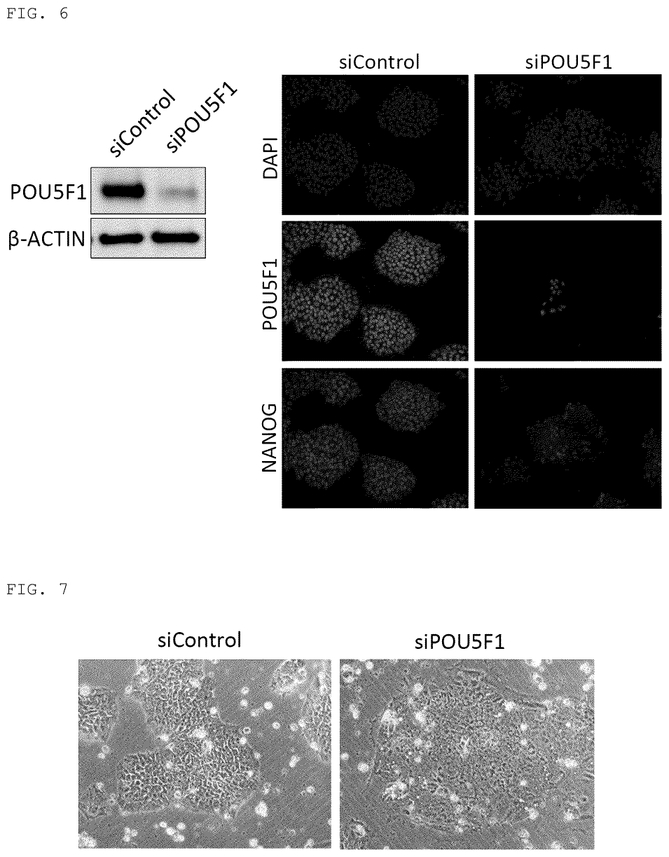

Effects of siRNA (siPOU5F1) on POU5F1 in human ES cells were confirmed by an immunoblotting method and an immunostaining method (FIG. 6). Cells on Day 3 after siPOU5F1 transfection were analyzed, and as a result, it was confirmed that the POU5F1 protein was significantly decreased as compared to cells transfected with negative control siRNA (scramble siRNA sequence against POU5F1: siControl) (FIG. 6). The siPOU5F1 transfection decreased an NANOG protein serving as a molecular marker for pluripotent stem cells.

Thus, the disappearance of pluripotency by the siPOU5F1 transfection was confirmed.

(Confirmation of Morphological Change of Human ES Cells by siPOU5F1 Transfection)

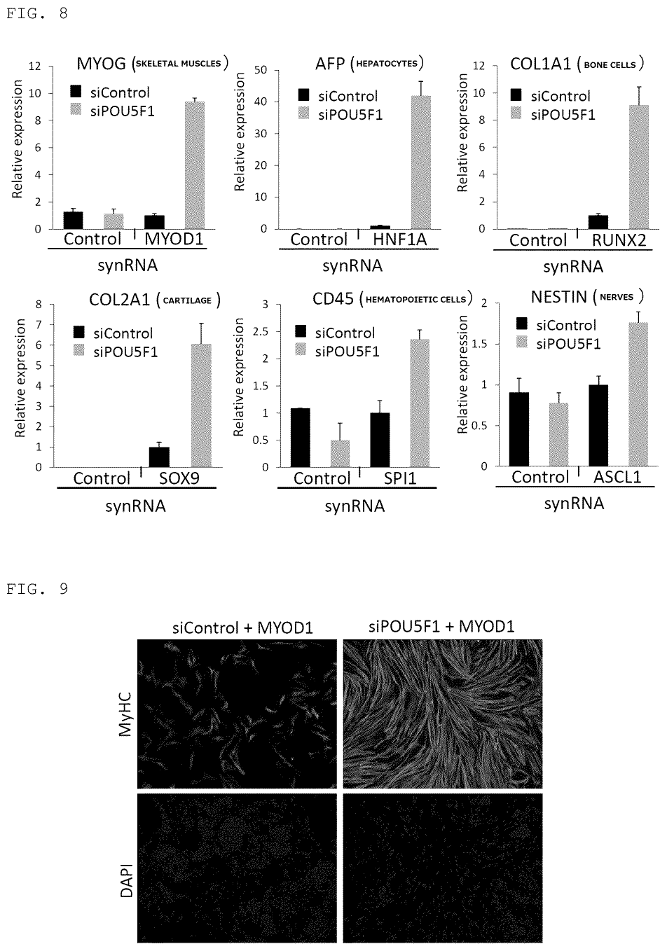

Human ES cells have small round shapes when their undifferentiated state is maintained. It was confirmed that cells underwent a morphological change into flat shapes when transfected with siPOU5F1, and were directed toward differentiation through POU5F1 suppression (FIG. 7).

As this Example's result, it was confirmed that pluripotent stem cells in which the expression amount of the POU5F1 protein had been reduced were able to be generated, and that the cells were promoted toward differentiation.

### Example 3

(Confirmation of Induction of Differentiation of Pluripotent Stem Cells in which Expression Amount of POU5F1 Protein has been Reduced into Desired Cell Types)

In this Example, it was confirmed whether differentiation into desired cell types was induced by introducing (adding) transcription factors required for induction of differentiation into the desired cell types to pluripotent stem cells in which the expression amount of the POU5F1 protein had been reduced. The details are as described below.

siPOU5F1 and modified synthetic RNAs (synRNAs) for a tissue-specific transcription factors were introduced into human ES cells, and the expression of differentiation marker genes MYOG (skeletal muscles), AFP (hepatocytes), COL1A1 (bone cells), COL2A1 (cartilage), CD45 (hematopoietic cells), and NESTIN (nerves) was examined by a real-time RT-PCR method.

As the tissue-specific transcription factors, MYOD1 (skeletal muscles), HNF1A (liver), RUNX2 (bone cells), SOX9 (cartilage), SPI1 (blood), and ASCL1 (nerves) were used. As a negative control, synRNA for mCherry or Emerald was introduced.

siControl or siPOU5F1 and synRNA were simultaneously introduced (added) to the cells, and 1 day after that, the synRNA was further introduced thereto twice. The next day (2 days after the first introduction), the cells were sampled and analyzed. The medium used was a medium for undifferentiated state maintenance. As a result, it was confirmed that, when siPOU5F1 was introduced in combination with the transcription factors, the expression of the differentiation markers for the respective tissues was significantly increased (FIG. 8). While, as siControl or the transcription factors were introduced alone, any increases in expression of the differentiation markers were hardly detected (FIG. 8).

As this Example's result, it was confirmed that differentiation into desired cell types was efficiently induced by introducing (adding) the transcription factors required for induction of differentiation into the desired cell types to the pluripotent stem cells in which the expression amount of the POU5F1 protein had been reduced.

### Example 4

(Confirmation of Differentiation of Pluripotent Stem Cells in which Expression Amount of POU5F1 Protein has been Reduced into Desired Cell Types)

In this Example, as examples of induction of differentiation into desired cell types, a myogenic differentiation (skeletal muscle cell differentiation) model using a myogenesis-regulating master transcription factor MYOD1, and a hepatocyte differentiation model using hepatocyte nuclear factor-1-alpha (HNF1A) were adopted. It is known that forced expression of MYOD1 alone cannot cause sufficient epigenetic changes and transcriptional changes in hESCs, resulting in poor myogenic conversion (see Cell reports 3, 661-670 (2013)).

### (Skeletal Muscle Cell Differentiation)

Human ES cells were co-transfected with siControl or siPOU5F1 and synRNA for MYOD1 (MYOD1-synRNA), and 1 day after that, were further transfected twice with MYOD1-synRNA. In this case, the cells were cultured in a skeletal muscle differentiation medium.

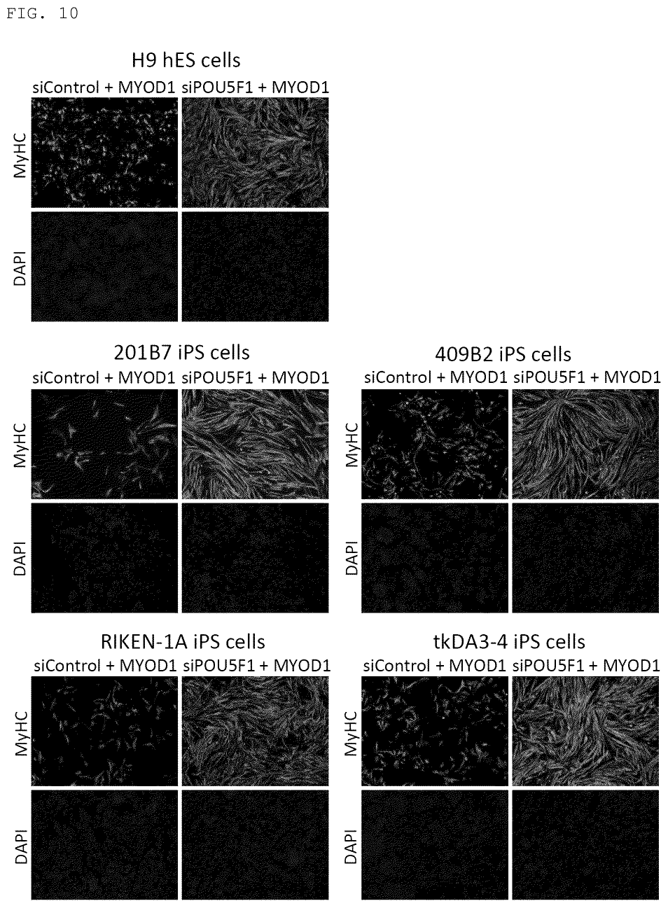

Two days after the last synRNA transfection, which was performed 2 days after the first transfection, the cells were fixed and immunostained. The ES cells transfected with siControl hardly underwent a morphological change even when transfected with MYOD1-synRNA, and few of the cells expressed Myosin Heavy Chain (MyHC) serving as a terminal differentiation marker for a skeletal muscle (FIG. 9).

However, as the ES cells transfected with siPOU5F1 were used, 2 days after the last MYOD1-synRNA transfection, fibrous muscle cells appeared at a high frequency and most of the cells were MyHC-positive, confirming that the cells were differentiated into skeletal muscle cells (FIG. 9).

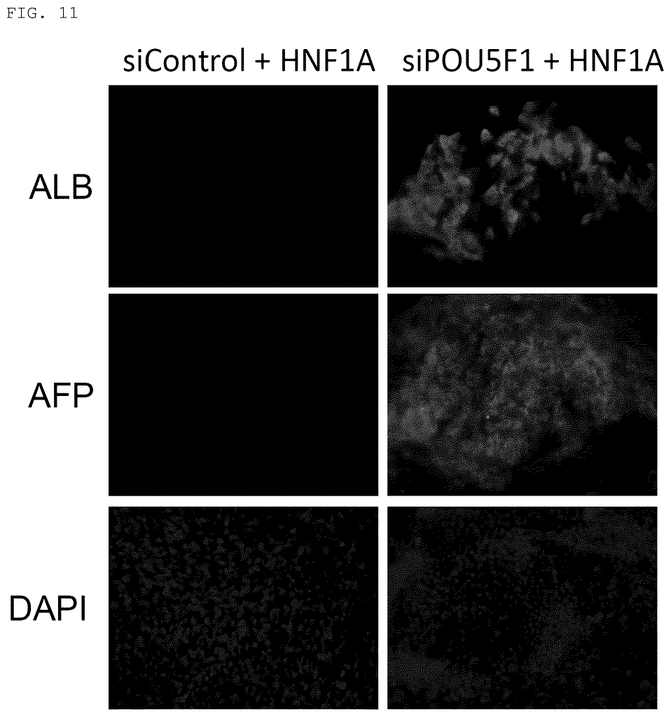

In addition, similar effects were also able to be obtained in another ES cell line (H9 hES cell) and a plurality of iPS cell lines (201B7 iPS cell, 409B2 iPS cell, RIKEN-1A iPS cell, and tkDA3-4 iPS cell) (FIG. 10).

### (Hepatocyte Differentiation)

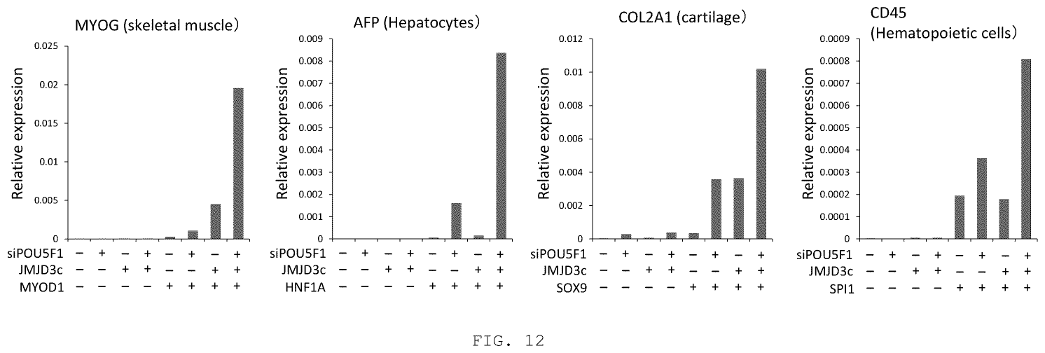

Human ES cells were co-transfected with siControl or siPOU5F1 and synRNA for HNF1A (HNF1A-synRNA), and 1 day after that, were further transfected twice with HNF1A-synRNA. From the day after the last synRNA transfection, which was performed 2 days after the first transfection, the cells were cultured in a hepatocyte differentiation medium, and 13 days later, the cells were fixed and immunostained. While, the ES cells transfected with siControl were transfected with HNF1A-synRNA, cells expressing AFP and albumin (ALB) serving as differentiation markers for hepatocytes were hardly observed (FIG. 11). Beside, in the case of the cells co-transfected with siPOU5F1 and HNF1A-synRNA, many cells expressing albumin and AFP were observed, and thus it was confirmed that the cells were differentiated into hepatocytes (FIG. 11).

As this Example's result, it was confirmed that differentiation into desired cell types was able to be efficiently induced by introducing the transcription factors required for induction of differentiation into the desired cell types into the pluripotent stem cells in which the expression amount of the POU5F1 protein had been reduced.

### Example 5

(Confirmation of Induction of Differentiation of Pluripotent Stem Cells in which Expression Amount of POU5F1 Protein has been Reduced and Demethylase is Forcibly Expressed into Desired Cell Types)

The inventors of the present invention have confirmed that pluripotent stem cells in which a demethylase is forcibly expressed allow the differentiation resistance of human pluripotent stem cells to disappear, thereby facilitating differentiation induction. A molecular mechanism by which the expression amount of the POU5F1 protein is reduced is different from a molecular mechanism by which the demethylase is forcibly expressed.

In view of the foregoing, it was confirmed whether differentiation into desired cell types was induced by introducing (adding) transcription factors required for induction of differentiation into the desired cell types to pluripotent stem cells in which the expression amount of the POU5F1 protein had been reduced and the demethylase was forcibly expressed, which were obtained by combining the reduction of the expression amount of the POU5F1 protein and the forced expression of the demethylase. The details are as described below.

Differentiation into the desired cell types was induced using synRNAs for tissue-specific transcription factors MYOD1, HNF1A, SOX9, and SPI1.

It was confirmed that the expression of differentiation marker genes MYOG (skeletal muscle), AFP (hepatocyte), COL2A1 (cartilage), CD45 (hematopoietic cell) was more increased in a synergistic manner in the case of differentiation performed by combining the reduction of the expression amount of the POU5F1 protein and the forced expression of the demethylase than in the case of differentiation performed by only one of the reduction of the expression amount of the POU5F1 protein or the forced expression of the demetylase, and the expression with synRNA (FIG. 12).

More specifically, an about 18.2-fold increase in expression of MYOG, an about 5.2-fold increase in expression of AFP, an about 2.8-fold increase in expression of COL2A1, and an about 2.2-fold increase in expression of CD45 were achieved by combining the reduction of the expression amount of the POU5F1 protein and the forced expression of the demethylase as compared to the case in which the expression amount of the POU5F1 protein was only reduced.

Further, the combination of the reduction of the expression amount of the POU5F1 protein and the forced expression of the demethylase achieved an about 4.3-fold increase in expression of MYOG, an about 54.0-fold increase in expression of AFP, an about 2.8-fold increase in expression of COL2A1, and an about 4.5-fold increase in expression of CD45 as compared to the case in which the demethylase was forcibly expressed.

In addition, in hepatocyte differentiation induction, the reduction of the expression amount of the POU5F1 protein achieved expression about 10.4 times as high as that achieved by the forced expression of the demethylase. Thus, concerning hepatocyte differentiation induction, the reduction of the expression amount of the POU5F1 protein showed a significant effect as compared to the forced expression of the demethylase.

As this Example's result, it was confirmed that the pluripotent stem cells in which the expression amount of the POU5F1 protein had been reduced and the demethylase was forcibly expressed had a synergistic differentiation induction effect (advantageous effect) as compared to the pluripotent stem cells in which the expression amount of the POU5F1 protein had been reduced and the pluripotent stem cells in which the demethylase was forcibly expressed.

### Example 6

(Examples of Differentiation into Desired Cell Types Using Pluripotent Stem Cells of the Present Disclosure)

In this Example, differentiation into various desired cell types was confirmed using pluripotent stem cells in which the expression amount of the POU5F1 protein had been substantially removed or reduced on the basis of the example described as Example 3.

(Differentiation into Skeletal Muscle Cells)

During 4-day culture, human pluripotent stem cells were co-transfected with siPOU5F1 and a MYOD1 gene (SEQ ID NO: 33, SEQ ID NO: 34) once, and then transfected with the MYOD1 gene three times. It was confirmed that the cells were differentiated into skeletal muscle cells through the 4-day culture.

(Differentiation into Hepatocytes)

During 12-day culture, human pluripotent stem cells were co-transfected with siPOU5F1 and a HNF1A gene (SEQ ID NO: 35, SEQ ID NO: 36) once, and then transfected with the HNF1A gene twice. It was confirmed that the cells were differentiated into hepatocytes through the 12-day culture.

(Differentiation into Neurons)

During 5-day culture, human pluripotent stem cells are transfected with siPOU5F1 once, and then transfected with a NEUROG1 gene (SEQ ID NO: 37, SEQ ID NO: 38), a NEUROG2 gene (SEQ ID NO: 39, SEQ ID NO: 40), a NEUROG3 gene (SEQ ID NO: 41, SEQ ID NO: 42), a NEUROD1 gene (SEQ ID NO: 43, SEQ ID NO: 44), and a NEUROD2 gene (SEQ ID NO: 45, SEQ ID NO: 46) three times. The cells can be differentiated into neurons through the 5-day culture.

### Example 7

(Examples of Differentiation into Desired Cell Types Using Pluripotent Stem Cells of the Present Disclosure by Use of Sendai Virus Vector)

In this Example, unlike Example 3, differentiation into various desired cell types is confirmed using pluripotent stem cells in which the expression amount of the POU5F1 protein has been substantially removed or reduced, through use of a Sendai virus vector instead of synthetic modified mRNA.

(Differentiation into Skeletal Muscle Cells)

A Sendai virus vector known per se (which is active at 33° C. and is inactivated at 37° C.) into which a transcription factor MYOD1 gene (SEQ ID NO: 33, SEQ ID NO: 34) has been cloned is used.

A suspension of human ES cells or iPS cells is infected with the Sendai virus vector at a multiplicity of infection (MOI) of 1 to 100 (25). After that, the cells are transferred to a culture plate, and the cells are cultured in a CO₂ incubator kept at 33° C. for 3 days. After that, the cells are transferred to a CO₂ incubator at 37° C., and culture is continued. As a result, skeletal muscle cells can be observed as differentiated cells.

(Differentiation into Hepatocytes)

A Sendai virus vector known per se into which a transcription factor HNF1A gene (SEQ ID NO: 35, SEQ ID NO: 36) has been cloned is used.

A suspension of human ES cells or iPS cells is infected with the Sendai virus vector at a MOI of 1 to 100 (25). After that, the cells are transferred to a culture plate, and the cells are cultured in a CO₂ incubator kept at 33° C. for 3 days. After that, the cells are transferred to a CO₂ incubator at 37° C., and culture is continued. As a result, hepatocytes can be observed as differentiated cells.

(Differentiation into Neurons)

A Sendai virus vector known per se into which a transcription factor NEUROG3 gene (SEQ ID NO: 41, SEQ ID NO: 42) has been cloned is used.

A suspension of human ES cells or iPS cells is infected with the Sendai virus vector at a MOI of 1 to 100 (25). After that, the cells are transferred to a culture plate, and the cells are cultured in a CO₂ incubator kept at 33° C. for 3 days. After that, the cells are transferred to a CO₂ incubator at 37° C., and culture is continued. As a result, neurons (motor neurons) can be observed as differentiated cells.

### CONCLUSION

The inventors of the present invention have confirmed by the above-mentioned Examples that the method of differentiating a pluripotent stem cell into a desired cell type with high efficiency and the differentiation induction kit for differentiating a pluripotent stem cell into a desired cell type with high efficiency of the present invention each have at least any one of the following effects.
(1) The period of time required for cell differentiation starting with the pluripotent stem cell is shortened and/or the differentiation induction efficiency is improved.
(2) As modified synthetic mRNA for a gene is used to introduce the gene into the pluripotent stem cell, the introduced gene is not incorporated into the genome of the pluripotent stem cell, with the result that there is no risk of canceration or the like after cell differentiation induction.
(3) In the introduction of the gene into the pluripotent stem cell using the modified synthetic mRNA, the timing and number of times of the addition of the mRNA for the gene can be easily changed, and hence optimal conditions specific to each of various desired cell types can be selected so as to differentiate the pluripotent stem cell into the desired cell types.
(4) A method of reducing undifferentiated state maintenance of a pluripotent stem cell and a method of reducing differentiation resistance thereof are combined with each other to shorten the period of time required for cell differentiation starting with the pluripotent stem cell and to improve the differentiation induction efficiency in a synergistic manner.

### INDUSTRIAL APPLICABILITY

According to the present invention, the novel method of differentiating a pluripotent stem cell into a desired cell type with high efficiency can be provided.

1-13. (canceled)

14. A method of differentiating a pluripotent stem cell into a desired cell type, comprising any one of the following steps (1) to (2):
(1) a step of adding an siRNA, shRNA, or antisense RNA having an action of substantially removing or reducing an expression amount of a POU5F1 protein, a demethylase gene, and a modified mRNA encoding a transcription factor required for induction of differentiation into the desired cell type to a pluripotent stem cell; and
(2) a step of adding a modified mRNA encoding a transcription factor required for induction of differentiation into the desired cell type and an siRNA, shRNA, or antisense RNA having an action of substantially removing or reducing an expression amount of a POU5F1 protein to a pluripotent stem cell which has a histone in which H3K27me3 modification has been substantially removed or reduced,
wherein the differentiation method is of the pluripotent stem cell into a hematopoietic cell, and the transcription factor of each of the steps (1) to (2) comprises SPI1, and the removing or reducing of the expression amount of the POU5F1 protein of each of the steps (1) to (2), and production of SPI1 protein of each of the steps (1) to (2) lead to the differentiation of the pluripotent stem cell and expression of markers of hematopoietic cell differentiation.

15. The differentiation method according to claim 14, wherein the pluripotent stem cell is co-transfected with siPOU5F1 and synRNA for SPI1.
